(19) United States
(12) Patent Application Publication
(10) Pub. No.: US 2012/0115778 A1
(43) Pub. Date: May 10, 2012

(54) Methods of Suppressing Appetite by the Administration of Antagonists of the Serotonin HTR1a or HTR2b Receptors or Inhibitors of TPH2

(75) Inventors: Gerard Karsenty, New York, NY (US); Vijay Yadav, Hinxton (GB); Franck Oury, New York, NY (US)

(73) Assignee: The Trustees of Columbia University in the City of New York, New York City, NY (US)

(21) Appl. No.: 13/383,050

(22) PCT Filed: Jul. 15, 2010
(86) PCT No.: PCT/US2010/042204

§ 371 Date: Jan. 9, 2012

Related U.S. Application Data
(60) Provisional application No. 61/225,754, filed on Jul. 15, 2009.

(51) Int. Cl. A61K 31/496; A61K 31/353; A61K 31/438; A61K 31/495; A61K 31/4045; A61P 19/00; A61K 31/404; A61K 31/473; A61K 38/22; A61P 3/00; A61P 3/04; A61K 31/4365; A61K 31/382

(52) U.S. Cl. 514/5.3; 514/291; 514/456; 514/253.01; 514/253.12; 514/278; 514/255.03; 514/254.04; 514/253.11; 514/419; 514/254.11; 514/432; 514/254.06; 514/252.13; 514/415; 514/253.07; 514/254.09; 514/297

(57) ABSTRACT

Methods for treating eating disorders associated with excessive weight gain, suppressing appetite, reducing body weight, or treating obesity in an animal by administering one or more antagonists of the serotonin Htr1a or Htr2b receptor, or a Tph2 inhibitor are provided, or combinations thereof.

### CROSS-REFERENCE TO RELATED APPLICATIONS

This application claims benefit of U.S. Provisional Appln. 61/225,754, filed Jul. 15, 2009, the entire contents of which are hereby incorporated by reference as if fully set forth herein, under 35 U.S.C. §119(e).

### STATEMENT OF GOVERNMENT INTEREST

This invention was made with Government support under NIH-RO1 DK58883. The Government has certain rights in the invention.

### FIELD OF THE INVENTION

The invention is in the field of treatment of body weight disorders, e.g., the suppression of appetite for the control of obesity.

### BACKGROUND OF THE INVENTION

The control of body weight is a complex process that is influenced by appetite, food ingestion, and energy expenditure. A number of mediators are known to be involved in the control of body weight and include hormones and cytokines such as leptin, ghrelin, melanocortin, agouti-related peptide, and neuropeptide Y (NPY). Normal weight control is important to good health and the lack of normal weight control represents a serious medical problem. Obesity is nearing epidemic levels in the United States and many other nations in the developed world (Mokdad et al., 2000, JAMA 291:1238-11245). The presence of obesity is strongly correlated with many medical problem, e.g., diabetes, hypertension, coronary artery disease (Kopelman, 2000, Nature 404:635-643).

Leptin is an adipocyte-derived hormone that regulates a broad spectrum of homeostatic functions, including appetite and energy expenditure, following its binding to the signaling form of its receptor, ObRb, present on neurons of the central nervous system (Friedman & Halaas, 1998, Nature 395:763-770; Spiegelman & Flier, 2001, Cell 104:531-543).

In addition to its effects on appetite and energy expenditure, one homeostatic function regulated by leptin in rodents, sheep and humans is bone remodeling, the mechanism whereby vertebrates renew their bones during adulthood (Karsenty, 2006, Cell Metab. 4:341-348; Pogoda et al., 2006, J. Bone Miner. Res. 21:1591-1599). Leptin regulates, exclusively through a neuronal relay, both phases of this process, resorption and formation (Ducy et al., 2000, Cell 100:197-207; Shi et al., 2008, Proc. Natl. Acad. Sci. USA 105:20529-20533). One mediator linking leptin signaling in the brain to bone remodeling is the sympathetic tone, which inhibits bone formation and favors bone resorption through the β adrenergic receptor (Adrβ2) expressed in osteoblasts (Elefteriou et al., 2005, Nature 434:514-520; Takeda et al., 2002, Cell 111:305-317). Hence, sympathetic activity can be used as a readout of leptin regulation of bone mass.

Serotonin is an indoleamine produced in enterochromaffin cells of the duodenum and in serotonergic neurons of brainstem that does not cross the blood brain barrier (Mann et al., 1992, Arch. Gen. Psychiatry 49:442-446). Thus, it is a molecule with two distinct functional identities, depending on its site of synthesis: a hormone when made in the gut and a neurotransmitter when made in the brain (Walther et al., 2003, Science 299:76; Yadav et al., 2008, Cell 135:825-837).

Serotonin is generated through an enzymatic cascade in which L-tryptophan is converted into L-5-hydroxytryptophan by an enzyme called tryptophan hydroxylase (Tph). This intermediate product is then converted to serotonin by an aromatic L-amino acid decarboxylase. There are two Tph encoding genes, Tph1 and Tph2, which are 71% identical in amino acid sequence and about 90% similar in the catalytic domain. While Tph1 controls serotonin synthesis in the periphery, Tph2 is responsible for serotonin synthesis in the brain (Walther et al., 2003, Science 299:76). The Tph enzymes are the rate limiting enzymes for the production of serotonin in either location.

There are currently 14 known serotonin receptor subtypes, classified into 7 families (5-HT1 to 5-HT7) based upon such factors as structure, function, and signal transduction properties (Hoyer et al., 2002, Pharmacol. Biochem. Behay. 71:533-554). 5-HT1a, 5-HT2b, and 5-HT2c receptors have received attention in connection with food intake and control of body weight (Vickers & Dourish, 2004, Curr. Opin. Investigational Drugs 5:377-388). Certain aspects of the serotonergic receptor system have been targeted for the treatment of obesity (Garfield & Heisler, 2009, J. Physiol. 587:49-60).

### SUMMARY OF THE INVENTION

The present invention provides methods of treating eating disorders associated with excessive weight gain, suppressing appetite, reducing body weight, or treating obesity in a patient, preferably mammals, and most preferably humans, by the administration of a therapeutically effective amount of an antagonist of the serotonin Htr1a receptor or an antagonist of the serotonin Htr2b receptor, including derivatives, analogs and variants thereof, or combinations thereof. The serotonin antagonists and agonists described herein can be specific or non-specific. Certain embodiments of this method further include administering an amount of an Htr2c agonist that increases or maintains the patient's bone mass. Other related embodiments further include administering an amount of leptin or a leptin receptor agonist, or analogs, derivatives or variants thereof to treat the patient.

In certain embodiments for treating eating disorders associated with excessive weight gain, suppressing appetite, reducing body weight, or treating obesity in a patient, a therapeutically effective amount of an inhibitor of Tph2 is administered, either alone or together with one or more antagonists of the Htr1a or the Htr2b receptors. Certain embodiments of this method further include administering an amount of an Htr2c agonist that increases or maintains the patient's bone mass.

In certain embodiments for treating eating disorders associated with excessive weight gain, suppressing appetite, reducing body weight, or treating obesity in a patient, treatment results in a reduction of the body weight of at least 2 kg, at least 5 kg, at least 10 kg, at least 15 kg, or at least 20 kg; or a reduction of the body weight of the patient of at least 3%, 5%, 10%, 15%, or 20%.

A method for decreasing the weight gain in a patient taking an agent selected from the group comprising tricyclic antidepressants selected from the group comprising amitriptyline, imipramine, doxepine; selective serotonin reuptake inhibitors selected from the group comprising paroxetine and fluoxetine; irreversible monoamine oxidase selected from the group comprising phenelzine, isocarboxazid, tranylcypromine, and steroids, comprising administering one or more Htr1a receptor antagonists, Htr2b receptor antagonists, or Tph2 inhibitors or combinations thereof including analogs, derivatives or variants thereof in amounts that decrease the weight gained by the patient while taking the agent.

Other embodiments are directed to methods of treating an eating disorder associated with excessive weight loss, increasing appetite or increasing body weight in a patient in need of such treatment, by administering to the patient a therapeutically effective amount of one or more Htr1a receptor agonists, Htr2b receptor agonists, or analogs, derivatives or variants thereof, or combinations thereof. The eating disorders include bulimia and anorexia. Certain embodiments of this method further include administering an amount of an Htr2c antagonist that increases or maintains the patient's bone mass. In certain embodiments, the methods result in an increase of the body weight of at least 2 kg, at least 5 kg, at least 10 kg, at least 15 kg, or at least 20 kg; or an increase of the body weight of the patient of at least 3%, 5%, 10%, 15%, or 20%. In certain embodiments the method further includes administering a leptin antagonist or derivatives, analogs or variants thereof.

Other embodiments include a method for achieving a desired level of appetite and bone mass in a patient, comprising administering one or more Htr1a or Htr2b receptor antagonists or agonists, or Tph1 inhibitor or Htr2c antagonists or agonists in respective amounts that achieve the desired levels of appetite and bone mass. This method can further include administering an amount of leptin or a leptin receptor agonist or antagonist that achieves the desired levels of appetite and bone mass. Agents that increase the amount or the half life of Tph2 in the brain can also be administered to increase appetite.

A method for increasing bone mass accrual in a patient having lower than desired bone mass by administering a therapeutically effective amount of a leptin receptor blocker, alone or together with an Htr2c agonist.

### BRIEF DESCRIPTION OF THE DRAWINGS

FIG. 1. Generation of Tph2−/− mice. (A) β-Galactosidase staining in the mouse brain during embryonic (E12.5-18.5) development. A: Anterior; P: Posterior. (B) Localization of Tph2-expressing neurons in the Dorsal (DR; from Bregma −4.04 to −5.49), Median (MR; from Bregma −4.04 to −4.48) and Caudal raphe (CR; from Bregma −4.84 to −7.48) in coronal sections of a mouse brain. (C) Tph2 expression by in situ hybridization, β-galactosidase staining and co-immunolocalization in Tph2LacZ/+ mice. Arrowheads indicate Tph2/β-Gal double positive cells. (D) Real-time PCR (qPCR) analysis of Tph2 expression in tissues of WT mice. (E) qPCR analysis of Tph2 expression in brainstem (BS) and duodenum (Duod) of WT and Tph2−/− mice.

(F) HPLC analysis of serotonin levels in different regions of brain in WT, Tph2+/− and Tph2−/− mice. (G) Serum serotonin levels in WT, Tph2+/− and Tph2−/− mice. (H) Mean litter size, serum biochemistry and body length in WT, Tph2+/− and Tph2−/− mice (n is indicated in superscript above each value). All panels (except F) *P<0.05; **P<0.01 (Student's t test). Error bars, SEM. Panel F (One way ANOVA, Newman-Keuls test); Different letters on 2 or more bars indicate significant differences between the respective groups (P<0.05).

FIG. 2. Low bone mass in Tph2−/− mice. (A-B) Histological analysis of vertebrae (A) and long bones (B) of WT, Tph2+/− and Tph2−/− mice. Mineralized bone matrix is stained in black by von Kossa reagent. Histomorphometric parameters. BV/TV %, bone volume over trabecular volume; Nb.Ob/T.Ar., number of osteoblasts per trabecular area; BFR, bone formation rate; OcS/BS, osteoclast surface per bone surface. (C) BV/TV % analysis in WT and Tph2−/− mice at 4, 6, 8 and 12 weeks after birth. (D) Lower bone density in long bones of 12-week-old Tph2−/− mice by μCT analysis along with lower Tb.Th (trabecular thickness) and decreased connectivity density (Conn.D). (E) Serum Dpd levels in WT and Tph2−/− mice. All panels *P<0.05; **P<0.01 Error bars, SEM)

FIG. 3. Brain-derived serotonin inhibits sympathetic activity. (A-B) HPLC analysis of serotonin levels in different regions of brain and serum serotonin levels in WT and Tph1−/−; Tph2−/− mice. (C) Histomorphometric analysis of vertebrae of WT, Tph1−/−, Tph2−/− and Tph1−/−; Tph2−/− mice. (D) Epinephrine levels in WT, Tph2+/−, Tph2−/− and Tph1−/−; Tph2−/− mice. (E) qPCR analysis of Ucp1 expression in brown adipose tissue of WT, Tph2+/−, Tph2−/− and Tph1−/−; Tph2−/− mice. (F) Epinephrine levels in the urine of WT, Tph2−/− and Tph2−/−; Adrβ2+/− mice. (G) Histomorphometric analysis of vertebrae of WT, Tph2−/− and Tph2−/−; Adrβ2+/− mice. All panels (except D and E) *P<0.05; **P<0.01 (Student's t test). Error bars, SEM. Panel D and E (One way ANOVA, Newman-Keuls test); Different letters on 2 or more bars indicate significant differences between the respective groups (P<0.05).

FIG. 4. Serotonin promotes bone mass through Htr2c receptors in VMH. (A-C) Analysis of axonal projections emanating from the serotonergic neurons of the brainstem. Coronal sections through the Dorsal (DR), Median (MR) raphe and ventromedial hypothalamus (VMH) nuclei from Sert-Cre/Rosa26REcfp, mice identifying serotonergic neurons and their axonal projections to VMH neurons through Ecfp immunohistochemistry (A). Retrograde (B) and anterograde (C) Rhodamine dextran labeling (Rh-dextran) in Tph2LacZ/+ mice. Coronal sections through the brainstem and hypothalamus showing colocalization of β-galactosidase staining and Rh-dextran fluorescence. (D) qPCR analysis of serotonin receptor expression in hypothalamus. (E) Double fluorescence situ hybridization analysis of Htr2c expression with Pomc or Sf1 expression in anterior (Top panel) and posterior (Bottom panel) VMH and arcuate nuclei. The third ventricle is outlined by a white line. (F) Histomorphometric analysis of vertebrae of WT, Htr2c−/−, Htr2c+/−, Tph2+/− and Htr2c+/−; Tph2+/− mice. (G-H) qPCR analysis of Ucp1 expression in brown adipose tissue (G) and epinephrine levels in urine (H) in WT, Htr2c−/− and Htr2c_{SF1}+/+ mice. (I) Histomorphometric analysis of vertebrae of WT, Htr2c_{loxTB}−/− and Htr2c_{SF1}+/+ mice. (J) HPLC analysis of glutamate levels in hypothalamus of WT and Htr2c−/− mice All panels (except J) *P<0.05; **P<0.01 (Student's t test). Error bars, SEM. Panel J (One way ANOVA, Newman-Keuls test); Different letters on 2 or more bars indicate significant differences between the respective groups (P<0.05).

FIG. 5. Leptin inhibits bone mass accrual by inhibiting brain-derived serotonin synthesis. (A) In situ hybridization analysis and co-immunolocalization of ObRb expression in serotonergic neurons. (B-C) qPCR analysis of Tph2 expression (B) and brainstem serotonin content (C) at different ages in WT and ob/ob female mice. (D-E) qPCR analysis of Tph2 expression following intra-cerebroventricular (ICV) infusion of leptin at different doses (D) and at different time points (E) in WT mice. (F) Immunohistochemical analysis of STAT3 phosphorylation in the dorsal and median raphe following leptin ICV. Arrows indicate pSTAT3/β-Gal positive cells. (G-H) qPCR analysis of Tph2 expression (G) and brainstem serotonin content (H) in WT, ob/ob and ob/ob; Tph2+/− mice. (I) Histomorphometric analysis of vertebrae of ob/ob and ob/ob; Tph2+/− mice. (J) Representative traces of action potentials recorded from WT mice before, during and after the application of leptin (100 nM). R.M.P. −43.0 mV. (K-L) Analysis of serotonergic neuron action potential (AP) frequency in brainstem slices from WT (K) and ObRb_{SERT}−/− (L) mice. All panels (except D, E, G, H and K) *P<0.05; **P<0.01 (Student's t test). Error bars, SEM. Panels D, E, G, H and K (One way ANOVA, Newman-Keuls test); Different letters on 2 or more bars indicate significant differences between the respective groups (P<0.05).

FIG. 6. Serotonin promotes food intake through Htr1a and Htr2b receptors on arcuate neurons. (A-B) Fat pad weights (A) and food intake (B) in WT, Tph2+/− and Tph2−/− mice. (C-E) Energy expenditure in WT and Tph2−/− mice; measured by volume of oxygen consumption (V_{O2}) (C), activity (D) and Heat production (E). (F) Analysis of axonal projections emanating from the serotonergic neurons. Cross of Sert-Cre and Rosa26REcfp mice identified projections reaching arcuate (Arc) nuclei in the hypothalamus through Ecfp immunohistochemistry colocalized to molecular markers of arcuate neurons (Pomc-1 and Npy) by in situ hybridization. Retrograde Rhodamine dextran labeling of the arcuate neurons identified serotonergic neurons in the brainstem in Tph2LacZ/+ mice through colocalization of β-galactosidase staining and Rh-dextran fluorescence in serotonergic neurons of the brainstem. (G) In situ hybridization analysis of Htr1a, Htr2b in Pomc1-expressing arcuate neurons of the hypothalamus. 3V: third ventricle. (H-I) Food intake (H) and fat pad weights (I) in WT, Htr1a−/− and Htr2b_{POMC}−/− mice. (J) qPCR analysis of hypothalamic gene expression in WT, Htr1a−/− and Htr2b_{POMC}−/− mice. (K) Food intake in WT, Tph2−/− mice before and after Mc4r antagonist (HS014) administration. (L) cFos induction in paraventricular nucleus of hypothalamus in WT, Tph2−/− mice before and after acute administration Mc4r agonist (MTII). 3V: third ventricle. (M-O) Volume of oxygen consumption (M), fat pad weight (N) and food intake (O) in WT, ob/ob, ob/ob; Tph2+/− and ob/ob; Tph2−/− mice. All panels (except A-B, H-J and M-O) *P<0.05; **P<0.01 (Student's t test). Error bars, SEM. Panels A-B, H-J and M-O (One way ANOVA, Newman-Keuls test); Different letters on 2 or more bars indicate significant differences between the respective groups (P<0.05).

FIG. 7. ObRb expression in serotonergic neurons is necessary and sufficient for leptin regulation of bone mass accrual, appetite and energy expenditure. (A) Histomorphometric analysis (vertebrae) of +/+; Sf1-Cre, ObRb_{SF1}−/−, +/+; Pomc1-Cre, ObRb_{POMC}−/−, +/+; Sert-Cre and ObRb_{SERT}−/− mice. (B) qPCR analysis of Ucp1 expression in brown adipose tissue in WT, ObRb_{SF1}−/−, ObRb_{POMC}−/− and ObRb_{SERT-/- mice. WT refers to +/+}; Sf1-Cre, +/+; Pomc1-Cre or +/+; Sert-Cre. (C-F) Food intake (C) volume of oxygen consumption (D), activity (E) and fat pad weights (F) in WT, ObRb_{SF1}−/−, ObRb_{POMC}−/− and ObRb_{SERT}−/− mice. (G) Representative photomicrographs of WT, ObRb_{SF1}−/−, ObRb_{POMC}−/− and ObRb_{SF1}−/− mice. (H) Brainstem serotonin content in WT, ob/ob, ObRb_{SERT}−/− and ObRb_{SF1}−/− mice. (I) qPCR analysis in the hypothalamus in WT, ObRb_{SERT}−/− and ob/ob mice. (J) Diameter of Pomc-expressing cells in WT and ObRb_{SERT}−/− mice. (K). Adipocytes are in yellow; serotonergic neurons are in pink; VMH is in blue and arcuate is in green. All panels (except B-F and H-I) *P<0.05; **P<0.01, ***P<0.001 (Student's t test). Error bars, SEM. Panels B-F and H-I (One way ANOVA, Newman-Keuls test); Different letters on 2 or more bars indicate significant differences between the respective groups (P<0.05).

FIG. 8. Generation of Tph2-deficient mice (A) Targeting strategy for generating Tph2−/− mice through homologous recombination in embryonic stem (ES) cells. (B) β-galactosidase staining of different tissues of WT (left) and Tph2−/− (right) mice brain (a-e) [a: Cerebral cortex (dorsal view); b: Cerebral cortex (ventral view); c: Cerebellum; d and e: Brain stem]. Positive brain areas are highlighted with dotted yellow lines. (C) Schematic representation of locus of β-galactosidase-positive neurons (in blue) in adult mouse brain. DR: dorsal raphe; MR; median raphe; CR: caudal raphe (D) Characterization of Tph2 expression throughout the brain. Series of brain 20 μm cryosections (A-W) from Tph2/LacZ mice were stained with β-galactosidase at 37° C., counterstained in eosin, cleared and mounted in DPX. Pictures 1× and inset 5×. Tph2-positive neurons are in blue. Please note that only brain regions with positive labeling have been shown in the 5× magnification. Bregma locations of sections are indicated below each panel. (E) Serum levels of T4 and Corticosterone in Tph2−/− mice. Serum T4 and corticosterone were measured by radio-immunoassay in Tph2−/− mice following manufacturer's instructions (MP Biomedicals, Corticosterone: Cat#07-120102; T4: Cat#06B-254011).

FIG. 9. Bone mineralization is normal in Tph2−/− mice. Analysis of non-mineralized bone matrix in Tph2−/− mice. Osteoid surface/bone surface was measured as an indicator of bone mineralization using the Osteomeasure software. Data are presented as Mean±SEM. *p<0.05. Student's t test.

FIG. 10. (A) Changes in Norepinephrine levels in WT and Tph2−/− brain. HPLC analysis of brain norepinephrine levels in WT and Tph2−/− brain. *p<0.05 SEM. (B) S3B. Body weight and serum hormone levels in WT and Tph1−/−; Tph2−/− mice. Body weight analysis, serum T4 and corticosterone, plasma leptin and insulin, and body length in 3 month-old WT and Tph1−/−; Tph2−/− mice. Body weight curve and hormonal changes in Tph2−/− has been presented in FIG. 1H and S1E and S6H. Number of mice used for each of the analysis is indicated in superscript above each value.

FIG. 11. (A) Neuro-anatomical tracing: Surgical site of application for rhodamine dextran. Rhodamine dextran application sites for arcuate, VMH and median raphe application. Brain section of Tph2LacZ/+ mice (200 μm) showing rhodamine dextran application sites for arcuate nucleus (A), Ventro medial hypothalamus (B), and median raphe (C). White lines and arrows indicate the exact sites of surgical application of rhodamine dextran. Tph2-expressing neurons were revealed by β-galactosidase staining. VMH, DMH and Arc are outlined by dashed line in panels. (B) Retrograde rhodamine-dextran tracing. Rhodamine-dextran retrograde tracing. After arcuate (Top panel) and VMH (Bottom panel) application of rhodamine dextran in Tph2LacZ/+ mice brains, coronal sections (40-50 μm) were prepared through the dorsal raphe (Top panel) and median raphe (Bottom panel) and stained with β-galactosidase or visualized for rhodamine dextran, demonstrating that these neurons project respectively to the arcuate and VMH neurons. Tph2-expressing neurons are revealed by β-galactosidase staining and rhodamine dextran images show the projections and cell body of the neurons. (C) In situ hybridization of Htr2c and Pomc1. Cross-sections through the VMH and arcuate (Arc) hypothalamus in WT mice. In situ hybridization analysis of Htr2c expression in VMH and arcuate nuclei in comparison to Sf1 and Pomc-1 expression on adjacent sections. (D)-(H) Analysis of Htr2c−/− mice. Food intake (D), Pgc1α expression in brown adipose tissue (E), fat pad weights (F) and body weight analysis (G) in WT and Htr2c−/− mice at indicated ages. Changes in body length plasma leptin, insulin and blood glucose levels in WT and Htr2c−/− mice at 3 months of age (H). (I) Western blot analysis of serotonin receptors in hypothalamus. 100 μg of hypothalamus lysate prepared from WT mice was electrophoresed on SDS-PAGE, blotted on PVDF/nitrocellulose membrane and probed with antibodies against Htr2c, Htr2b, Htr1a and actin. (J) Htr2c re-expression in mice. In situ hybridization analysis of Htr2c−/− and Htr2c re-expression in ventromedial hypothalamus using Sf1-Cre mice.

FIG. 12. Genetic interaction between leptin and serotonin. Real-time PCR analysis of Ucp1 expression in brown adipose tissue in WT, ob/ob and ob/ob; Tph2+/− mice at 3 months of age. p<0.05, SEM.

FIG. 13. (A)-(B) Glucose metabolism in Tph2−/− mice. Feeding blood glucose levels (A) Glucose tolerance (A) and insulin tolerance (B) tests in 3-month-old WT and Tph2−/− mice. (C) MTII-induced changes in cFos expression in WT and Tph2−/− hypothalamus. (D)-(G) Energy Expenditure analysis in Htr1a−/− and Htr2bPOMC−/− mice. Volume of O₂ consumption (D), locomoter activity (E), heat production (F) and Ucp1 expression (G) in WT, Htr1a−/− and Htr2bPOMC−/− mice. (H) Body weight curve in Tph2−/− mice. Body weight curve for Tph2 mice. WT, Tph2+/− and Tph2−/− mice were fed regular rodent chow and weighed at indicated time points. *p<0.05, **p<0.01, ***p<0.001 Error bars, SEM.

FIG. 14. (A)-(D) In situ hybridization analysis in ObRb deletion in different regions of brain. Specificity of Cre drivers and analysis of cell-specific deletion of leptin receptor (A-B). Coronal sections through dorsal and median raphe (DR and MR) nuclei, and ventromedial hypothalamus (VMH) and arcuate (ARC) nuclei (outlined by dashed lines) in adult mice. (A) β-galactosidase staining in Sert-Cre/Rosa26R, Sf1-Cre/Rosa26R and Pomc-Cre/Rosa26R mice. (B) In situ hybridization with ObRb probe in ObRbSERT−/−, ObRbSF1−/−, ObRbPOMC−/− mice. Epinephrine levels in the urine (C) and heat production (D) in WT, ObrbSF1−/−, ObrbPOMC−/−, ObrbSERT−/− and ob/ob mice. (E) Body weight curve for ObRb deletion in different nuclei in the brain. WT, +/+; Sf1-Cre, +/+; Pomc-Cre, +/+; Sert-Cre, ObrbSF1−/−, ObrbPOMC−/− and ObrbSERT−/− mice were fed regular rodent chow and weighed once a week. There was no significant difference in the body weights between WT, +/+; Sf1-Cre, +/+; Pomc-Cre and +/+; Sert-Cre mice. *p<0.05, **p<0.01, ***p<0.001 Error bars, SEM. (F)-(G) Glucose metabolism in ObRbSERT−/− mice. Feeding blood glucose levels (F) Glucose tolerance (F) and insulin tolerance (G) tests in 3-month-old WT and ObRbSERT−/− mice. (H) Serum levels of T4 and Corticosterone in ObRbSERT−/− mice. Serum T4 and corticosterone were measured by radio-immunoassay in ObRbSERT−/− mice following manufacturer's instructions (MP Biomedicals, Corticosterone: Cat#07-120102; T4: Cat#06B-254011). (I) Bone mineralization is normal in ObRbSERT−/− mice. Analysis of non-mineralized bone matrix in ObRbSERT−/− mice. Osteoid surface/bone surface was measured as an indicator of changes in bone mineralization using the osteomeasure software. Data are presented as Mean±SEM. *p<0.05. Student's t test.

FIG. 15. Changes in Cart and Tph2 expression brain. Real-time PCR analysis of Cart expression in hypothalamus in WT, ob/ob and ob/ob; Tph2+/− mice at 3 months of age (A). (B) Real-time PCR analysis of Tph2 expression in brainstem in WT and ObRbPOMC−/− mice. p<0.05, SEM.

FIG. 16. Graph of body mass index.

FIG. 17. (A) In situ hybridization of Htr1a and Pomc1. Cross-sections through the Ventromedial hypothalamus (VMH) and arcuate (Arc) hypothalamus in WT and Htr1a−/− mice. In situ hybridization of Htr1a expression in VMH and arcuate nuclei in comparison to Pomc-1 expression on adjacent sections. (B-C) Analysis of appetite of Htr1a_{Pomc}−/− mice. (B) Measurement of the food intake (g) within 12 hours and 24 hours and (C) body weight analysis in WT, Htr1a_{Pomc}+/− and Htr1a_{Pomc}−/− mice at 3 and 6 months of age. *(p<0.05, SEM). (D-E) Analysis of appetite in Htr1a; Htr2b_{Pomc}−/− mice. (D) Measurement of the food intake (g) in WT and Htr1a; Htr2b_{Pomc}−/− mice within 12 hours and 24 hours. (E) Food intake analysis in 24 hours (percentage) in Htr1a_{pomc}−/−, Htr2b_{Pomc}−/− and Htr1a; Htr2b_{Pomc}−/− mice in comparison to WT mice littermates. *(p<0.05, SEM). (F) Real-time PCR analysis of Mc4r, Pomc-1, Cart, Mch, Hypocretine and Npy expression in hypothalamus of WT and Htr1a; Htr2b_{Pomc}−/− mice. *(p<0.05, SEM).

FIG. 18. (A) Western blot analysis of CREB phosphorylation and CREB in hypothalamic explants treated with PBS, serotonin (50□M), Htr1a antagonist (LY426955) (50□M) or serotonin (50□M)+Htr1a antagonist (LY426955) (50□M). (B) Analysis of CREB phosphorylation by immunofluorescence using P-CREB (S133) antibody. Immunofluorescence was performed on coronal sections of wild type (WT) hypothalamic explants previously treated with, PBS, 50 □M serotonin, 50 □M Htr1a antagonist (LY426955) or 50 □M serotonin+50 □M Htr1a antagonist (LY426955) for 30 min. The first row represents large bright field images of hypothalamic sections and the immunofluorescence analysis of the restricted hypothalamic region containing the Arcuate (Arc) neurons. Ventromedial hypothalamus (VMH) and Arcuate (Arc) are outlined with dashed. (C-D) Analysis of the appetite of Creb_{Pomc}−/− mice. (C) Measurement of the food intake (g) within 12 hours and 24 hours and (D) body weight analysis in WT, Creb_{Pomc}+/− and Creb_{Pomc}−/− mice. *(p<0.05, SEM). (E) Real-time PCR analysis of Mc4r, Pomc-1, Cart, Mch, Hypocretine and Npy expression in hypothalamus of WT and Creb_{Pomc}−/− mice. *(p<0.05, SEM).

FIG. 19. (A) Molecular structure of Htr1a antagonist (LY426955). (B) Food intake analysis of WT mice after treatment with Htr1a antagonist (LY426955). Food intake analysis (g) was made in WT mice after daily injection of vehicle or Htr1a antagonist (LY426955) at different doses (5, 10, 20 mg/Kg of body weight) for 1 month. The measurements were performed within 12 hours, 24 hours and 36 hours. (C-D) Food intake analysis of leptin deficient mice (ob/ob). WT and ob/ob mice were daily injected during 1 months with vehicle or Htr1a antagonist (LY426955) at 20 mg/Kg of body weight. The measurements were made within 12 hours, 24 hours and 36 hours. *(p<0.05, SEM). (D-G) (D) Body weight analysis of WT and ob/ob mice daily injected with either vehicle or Htr1a antagonist (LY426955). (E) Body weight, (F) fat pad weight and (G) food intake analysis of WT and ob/ob mice after 1 month of daily injection with either vehicle or Htr1a antagonist (LY426955) *(p<0.05, SEM).

### DEFINITIONS

An “antagonist of a serotonin receptor,” as used herein, refers to a substance which reduces the action or effect of signaling through the serotonin receptor. Preferably, such reduction of the action or effect of the serotonin receptor occurs by a mechanism that involves binding of the substance to the serotonin receptor. Preferably, such reduction of the action or effect of the serotonin receptor results in the suppression of appetite in a mammal, preferably such that the body weight of the mammal is lowered. Antagonists of the Htr 1a, 2b and 2c receptors are discussed herein. An Htr-specific antagonist is one that does not significantly bind to or inactivate or reduce the activity of any other serotonin receptor, for example an Htr1a specific antagonist does not bind significantly to an Htr2b or Htr1c receptor. A non-specific antagonist is one that will significantly bind to or inactivate more than one serotonin receptor.

An “agonist of a serotonin receptor,” as used herein, refers to a substance which increases the action or effect of signaling through the serotonin receptor. Preferably, such increase of the action or effect of the serotonin receptor occurs by a mechanism that involves binding of the substance to the serotonin receptor. Preferably, such increase of the action or effect of the serotonin receptor results in the increase of appetite in a mammal, preferably such that the body weight of the mammal is raised. Agonists of the Htr 1a, 2b and 2c receptors are discussed herein. An Htr-specific agonist is one that does not significantly bind to any other serotonin receptor or significantly activate or increase activity of any other serotonin receptor, for example an Htr1a specific agonist does not bind significantly to an Htr2b or Htrlc receptor. A non-specific agonist is one that will significantly bind to or activate more than one serotonin receptor.

A “Tph2 inhibitor” is a substance that reduces the amount of 5-hydroxytryptophan produced from tryptophan by Tph2 in a suitable assay as compared to the amount of 5-hydroxytryptophan produced from tryptophan by Tph2 in the assay in the absence of the substance. Preferably, Tph2 inhibitors reduce the amount of 5-hydroxytryptophan produced from tryptophan by Tph2 by about 10%, about 20%, about 30%, about 40%, about 50%, about 60%, about 70%, about 80%, about 90%, or about 95%. In preferred embodiments, the Tph2 inhibitor inhibits Tph2 without significantly affecting the level of gut-derived serotonin. Methods of obtaining such inhibitors include screening for agents that inhibit Tph2 to a much greater extent than Tph1. Preferably, compounds that inhibit Tph2 to a much greater extent than Tph1 have an IC₅₀ for Tph1 that is at least about 10-fold, about 50-fold, or about 100-fold greater than their IC₅₀ for Tph2.

An antagonist of the serotonin Htr1a receptor, an antagonist of the serotonin Htr2b receptor, or a Tph2 inhibitor is said to be administered in a “therapeutically effective amount” if the amount administered results in a desired change in the physiology of the patient, e.g., results in a decrease in weight and/or suppression of appetite. An antagonist of the serotonin Htr1a receptor, an antagonist of the serotonin Htr2b receptor, or a Tph2 inhibitor is also said to be administered in a “therapeutically effective amount” if the amount administered enhances the therapeutic efficacy of another therapeutic agent. For example, if the amount of an Htr1a antagonist administered enhances the weight loss due to concomitant administration of another therapeutic agent used for weight-loss, e.g., sibutramine, then that amount of Htr1a antagonist is considered to be a therapeutically effective amount. The efficacy of treatment according to the methods of the present invention can be monitored by measuring changes in weight or food intake before and over time after treatment according to the methods of the present invention.

A “patient” is a mammal, preferably a human, but can also be companion animals such as dogs or cats, or farm animals such as horses, cattle, pigs, or sheep.

A patient “in need of treatment” by the methods of the present invention does not include a patient being treated with an Htr1a antagonist, an Htr2b antagonist, or a Tph2 inhibitor where the patient is being treated with the Htr1a antagonist, Htr2b antagonist, or Tph2 inhibitor for a purpose other than to suppress appetite and/or reduce body weight. Thus, a patient in need of treatment by the methods of the present invention does not include a patient being treated with an Htr1a antagonist, an Htr2b antagonist, or a Tph2 inhibitor for the purpose of treating anxiety, depression, psychosis, migraine, loss of memory, sexual dysfunction, hypertension, sleep disturbances, or as a neuroleptic or cognitive enhancer.

Accordingly, for the purposes of this invention, administering an Htr1a antagonist, an Htr2b antagonist, or a Tph2 inhibitor to a patient “in need of treatment” encompasses only those instances where it is known that the patient is obese or otherwise would benefit from suppression of appetite or a decrease in weight. Thus, such methods do not encompass administering to a patient who happens to be obese a therapeutically effective amount of an Htr1a antagonist, an Htr2b antagonist, or Tph2 inhibitor for a purpose other than to treat the obesity.

### DETAILED DESCRIPTION OF THE INVENTION

The present invention is based in part on the finding that leptin exerts its effects on decreasing appetite and increasing energy expenditure by inhibiting the synthesis and release of brain derived serotonin (BDS) in the brainstem, and that BDS increases appetite via serotonin Htr1a and Htr2b receptors on arcuate neurons in the hypothalamus.

The present invention provides methods of treating eating disorders associated with excessive weight gain, suppressing appetite, reducing body weight, or treating obesity by the administration of therapeutically effective amount of one or more serotonin Htr1a receptor antagonists, serotonin Htr2b receptor antagonists, Tph2 inhibitors or combinations thereof, including derivatives analogs and variants thereof, to a patient in need of such treatment. In certain embodiments the body weight is reduced by at least 2 kg, at least 5 kg, at least 10 kg, at least 15 kg, or at least 20 kg or a reduction of the body weight of the patient of at least 3%, 5%, 10%, 15%, or 20% is achieved. Other embodiments of the invention include treating eating disorders associated with excessive weight loss such as anorexia or bulimia, increasing appetite or body weight, by administering one or more agonists of the Htr1a or 2b receptor or combinations thereof, including derivatives analogs and variants thereof, to a patient in need of such treatment. In certain embodiments the body weight is increased by at least 2 kg, at least 5 kg, at least 10 kg, at least 15 kg, or at least 20 kg or an increase of the body weight of the patient of at least 3%, 5%, 10%, 15%, or 20% is achieved.

Agents that increase the amount or the half life of Tph2 in the brain can also be administered to increase appetite.

BDS is also increases bone mass through binding to the Htr2c receptor (International Patent Publication WO 2009/045900). The present invention is based in part on the unexpected observation that the effects of BDS on appetite and energy expenditure on the one hand, and bone mass on the other, are mediated by different serotonin receptors located in different portions of the hypothalamus. The knowledge of these different effects mediated by different receptors allows for the possibility of separately modulating the effects of BDS on appetite and bone mass by the appropriate choice of combination therapy with antagonists or agonists of the Htr1a, Htr2b and the Htr2c receptor. US Provisional Application Ser. No. 60/976,403, and International PCT Application WO 2009/045900.

For example, a combination of an agonist of the Htr1a or Htr2b receptors with an agonist of the Htr2c receptor would be expected to increase both appetite and bone mass. Accordingly, the present invention provides a method of increasing appetite and increasing bone mass in a patient in need of such treatment by the administration of an agonist of the Htr1a receptor or an agonist of the Htr2b receptor (or combinations thereof) and an agonist of the Htr2c receptor. Conversely, a combination of an agonist of the Htr1a or Htr2b receptors with an antagonist of the Htr2c receptor would be expected to stimulate appetite and decrease bone mass.

A combination of an antagonist of the Htr1a or Htr2b receptors with an agonist of the Htr2c receptor would be expected to suppress appetite and increase bone mass. Accordingly, the present invention provides a method of suppressing appetite and increasing bone mass in a patient in need of such treatment by the administration of an antagonist of the Htr1a receptor or an antagonist of the Htr2b receptor and an agonist of the Htr2c receptor. In certain other embodiments, doses of the agents are selected that result in the suppression of appetite while bone mass is either not affected (i.e., does not decrease) or increases.

Certain embodiments are directed to administering a therapeutically effective amount of a combination of an antagonist of the Htr1a or Htr2b receptors with an antagonist of the Htr2c receptor to suppress appetite and lower bone mass in a patient in need of such treatment.

In those embodiments of the present invention where an antagonist of the Htr2b receptor is employed, it is preferred that the antagonist of the Htr2b receptor is specific for Htr2b receptors in the brain and does not function as an antagonist of Htr2b receptors in the periphery. Action at peripheral Htr2b receptors is thought to underlie the cardiopathy exhibited by certain weight-loss drugs, such as fenfluramine (Fitzgerald et al., 2000, Mol. Pharmacol. 57:75-81). Agonists and antagonists of serotonin receptors for use in the present invention can be either specific or nonspecific.

Data presented herein show that leptin exerts its effects on appetite, energy expenditure, and bone mass by decreasing the synthesis and release of BDS. This finding allows for certain embodiments of the invention that combine treatment with leptin, leptin receptor agonists or leptin receptor antagonists, with antagonists or agonists of serotonin receptors Htr1a, Htr2b, or Htr2c in order to achieve a desired balance between effects on appetite and bone mass. Accordingly, the present invention provides a method of suppressing appetite and increasing or maintaining bone mass in a patient in need of such treatment by administering leptin or a leptin receptor agonist with an agonist of the Htr2c receptor. Leptin agonists include LEP-(116-130) or a synthetic peptide corresponding to the sequence (Ser-Cys-Ser-Leu-Pro-Gln-Thr), or an analog, variant or derivative thereof. Marina Rozhayskaya-Arena et al., Vol. 141, No. 7, Endocrinology; Design of a Synthetic Leptin Agonist: Effects on Energy Balance, Glucose Homeostasis, and Thermoregulation.

In embodiments of the present invention where a patient is administered more than one therapeutic agent, e.g., both an antagonist of the serotonin Htr1a receptor and an antagonist of the serotonin Htr2b receptor, the therapeutic agents may be administered together in a single pharmaceutical composition or separately, each in its own pharmaceutical composition. The frequency and amount of the therapeutic agent will vary.

In certain embodiments, the present invention provides methods where a patient is administered an antagonist of the serotonin Htr1a receptor, an antagonist of the serotonin Htr2b receptor, or a Tph2 inhibitor in combination with another active pharmaceutical ingredient where the other active pharmaceutical ingredient is administered for a purpose unrelated to controlling body weight but is known to have the undesirable side effect of increasing body weight. For example, an embodiment is directed to a method for decreasing the weight gain in a patient taking an agent selected from the group comprising tricyclic antidepressants, selective serotonin reuptake inhibitors, irreversible monoamine oxidase, and steroids, by administering an amount of an antagonist of the serotonin Htr1a or Htr2b receptors, a Tph2 inhibitor, or combinations thereof that decreases the weight gained by the patient while taking the agent. The use of the antagonist of the serotonin Htr1a receptor, the antagonist of the serotonin Htr2b receptor, or the Tph2 inhibitor in combination with the other active pharmaceutical ingredient will suppress appetite and/or decrease body weight, thus alleviating at least some of the undesirable effects of the other active pharmaceutical ingredient. Examples of such other active pharmaceutical ingredients include tricyclic antidepressants (e.g., amitriptyline, imipramine, doxepine), selective serotonin reuptake inhibitors (e.g., paroxetine, fluoxetine), irreversible monoamine oxidase inhibitors (e.g., phenelzine, isocarboxazid, tranylcypromine), and steroids (e.g., prednisone).

In certain embodiments, the methods of the present invention comprise the step of identifying a patient in need of therapy for obesity or suppression of appetite. Similar methods will identify patients who need stimulation of appetite to fight an eating disorder such as anorexia or bulimia, or lower than desired weight. Thus, the present invention provides a method of identifying and treating a patient for obesity or suppression of appetite comprising:

(a) identifying a patient in need of therapy for obesity or suppression of appetite;
(b) administering to the patient a therapeutically effective amount of an Htr1a antagonist, an Htr2b antagonist, or a Tph2 inhibitor.

In methods such as that described immediately above, “identifying a patient in need of therapy for obesity or suppression of appetite” refers to knowingly selecting for treatment such a patient. That is, such methods do not encompass administering to the patient a therapeutically effective amount of an Htr1a antagonist, an Htr2b antagonist, or Tph2 inhibitor where the patient is not selected for such administration because the patient is obese or otherwise would benefit from suppression of appetite. Thus, such methods do not encompass administering to a patient who happens to be obese a therapeutically effective amount of an Htr1a antagonist, an Htr2b antagonist, or Tph2 inhibitor for a purpose other than to treat the obesity; such methods encompass only the administration of an Htr1a antagonist, an Htr2b antagonist, or a Tph2 inhibitor for the purpose of treating obesity or suppressing appetite.

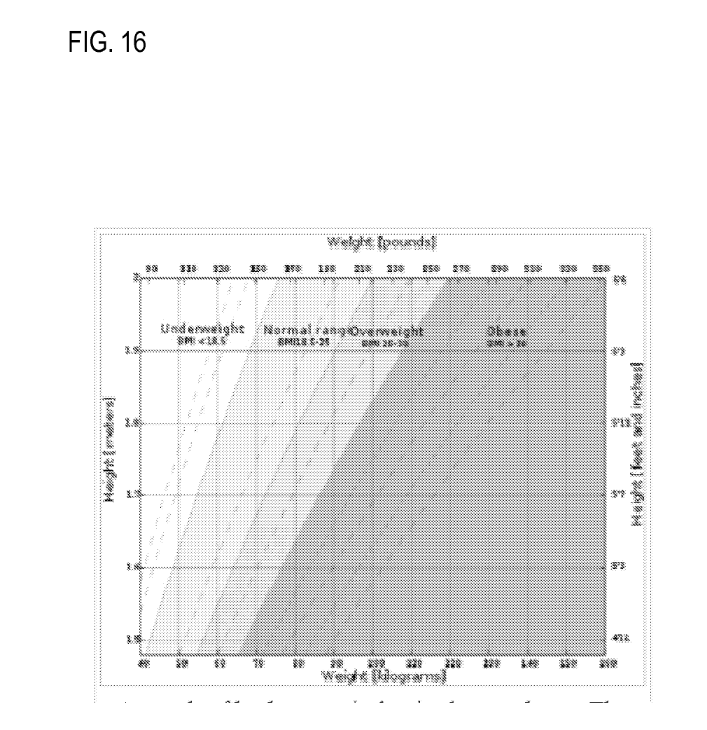

In certain embodiments, the patient has been selected for administration of an Htr1a antagonist, an Htr2b antagonist, or Tph2 inhibitor because the patient has been identified as being overweight (i.e., having a body mass index of from 23 to 27.5 kg/m²) or as being obese (i.e., having a body mass index of from 27.6 to 40 kg/m²). In certain embodiments, the patient has been identified as having a body mass index in the range indicated as “overweight” in the graph shown in FIG. 16 or as “obese” in the graph shown in FIG. 16.

### Results

Numerous studies in the last 16 years have aimed at drawing a precise map of the circuitry used by leptin signaling in the brain to fulfill these and other functions. Ducy et al., 2000, Cell 100:197-207; and Yadev et al., Cell 138:976-989, 2009). Following the lead provided by chemical lesion experiments and expression studies of the leptin receptor, these studies were most often based on the assumption that leptin signals in hypothalamic neurons to regulate appetite and energy expenditure (For review see Elmquist et al., 1999 NEURON 22: 221-232). Surprisingly, however, cell-specific deletion experiments of the leptin receptor in various hypothalamic neurons have failed to increase appetite or energy expenditure in mice fed a normal chow as leptin deficiency does (Dhillon et al., 2006, Neuron 49:191-203; Balthasar et al., 2004, Neuron 42:983-991). These data raised the prospect that leptin may act elsewhere in the brain to affect appetite.

The location of serotonergic neurons was defined according to Jensen et al (Jensen et al., 2008, Nat. Neurosci. 11, 417-419) as follows: dorsal raphe (B4, B6 and B7), median raphe (B5, B8 and B9) and caudal raphe (B1, B2 and B3) nuclei. Together these neurons will be referred herein as serotonergic neurons of the brainstem. Serotonin synthesis is initiated by hydroxylation of tryptophan, a rate-limiting reaction performed by the enzyme tryptophan hydroxylase 2 (Tph2) in the brain (Walther et al., 2003, Science 299:76).

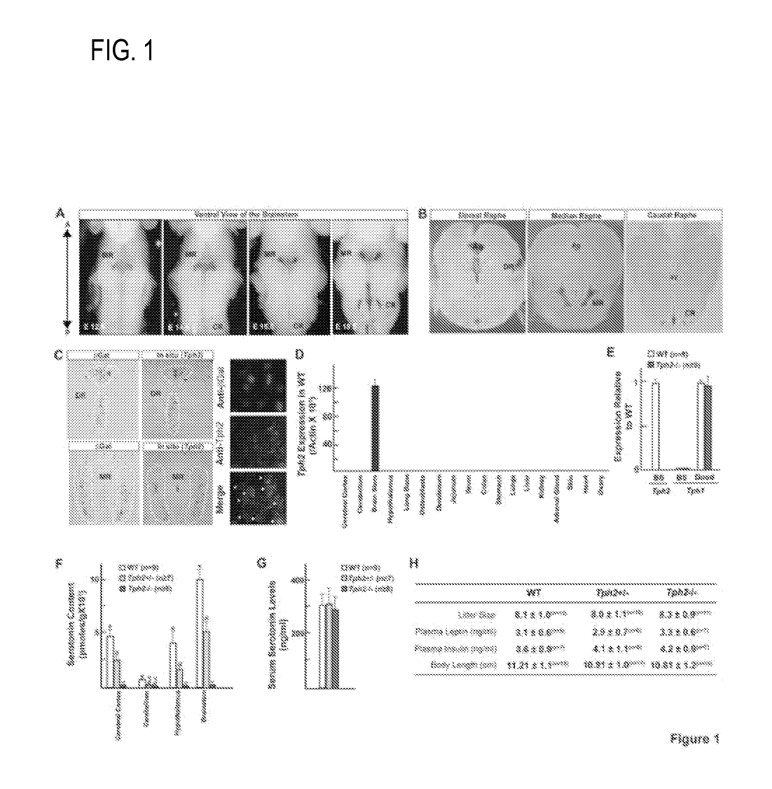

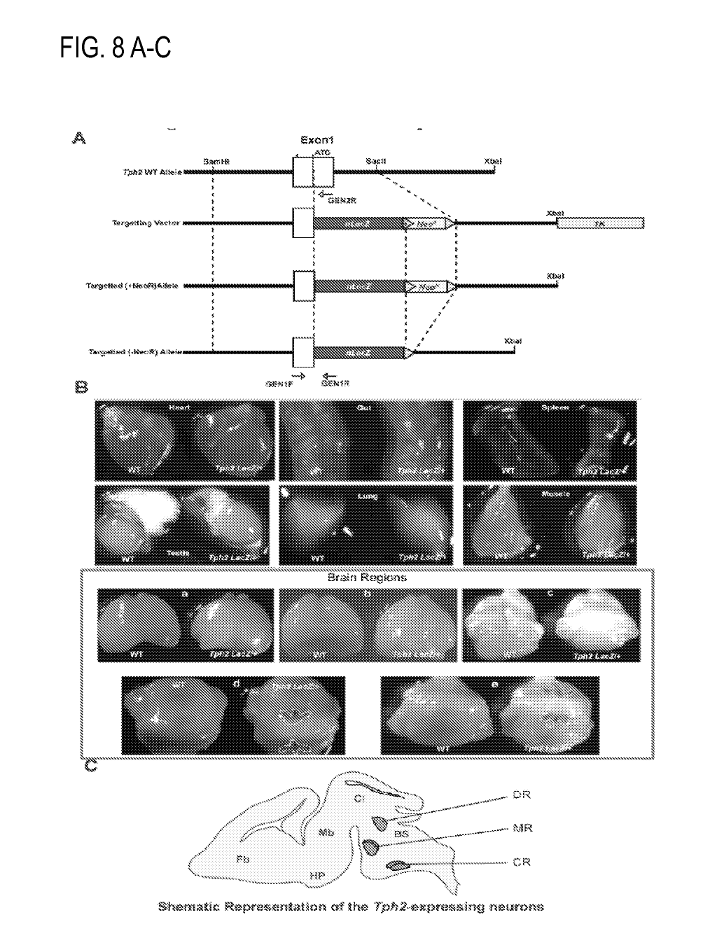

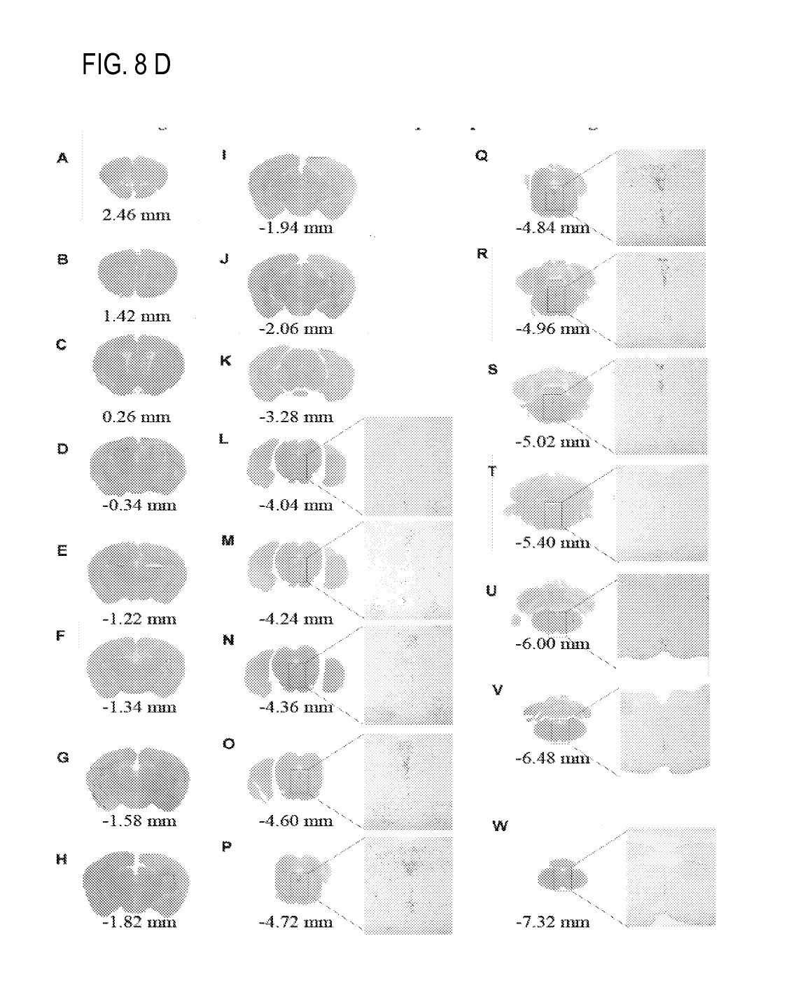

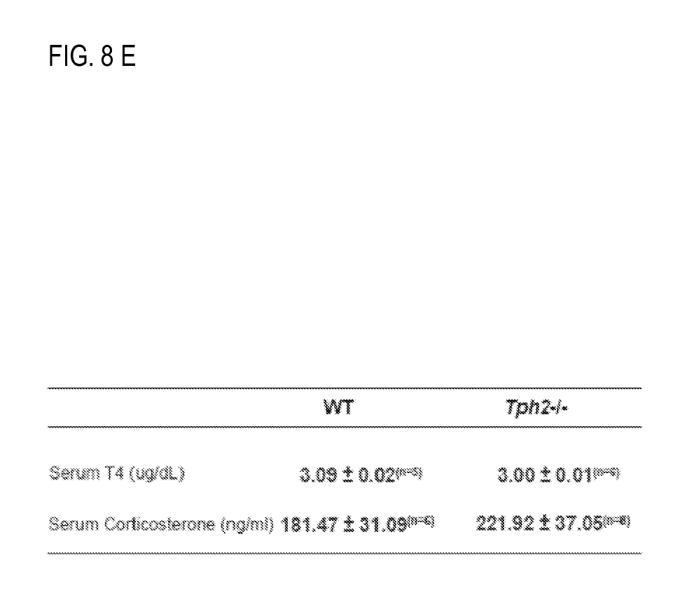

To study serotonergic cells in the brain, Tph2−/− mice were generated by disrupting Tph2 by inserting LacZ in its locus (FIG. 8A). β-galactosidase staining of the whole brain of Tph2−/− mice showed that during embryonic development Tph2 expression was detected as early as E12.5 in neurons of the dorsal and median raphe nuclei in the brainstem (FIG. 1A and data not shown). At E14.5, 15.5 and 18.5, (3-galactosidase staining was also detected in neurons of the caudal raphe nuclei of the brainstem (FIG. 1A-B) but not in other areas of the brain or in peripheral tissues (FIG. 8B-D). To determine whether 3-galactosidase staining is a faithful representation of Tph2 endogenous expression, in situ hybridization was performed and co-immunolocalization of Tph2 and β-galactosidase was demonstrated. These experiments revealed a tight concordance between Tph2 expression and β-galactosidase staining (FIG. 1C). After birth, Tph2 expression measured by real-time PCR was 4 orders of magnitude higher in the brainstem than in other parts of the brain or in peripheral tissues (FIG. 1D). Based on these criteria, Tph2 expression is specific to serotonergic neurons of the brainstem.

Tph2−/− mice were born at the expected Mendelian ratio, had a normal size and appearance, and were normally fertile (FIG. 1H and data not shown). The near complete absence of detectable serotonin in the brain of Tph2−/− mice verified that this gene had been successfully inactivated and was consistent with the fact that Tph1 expression in the brain was not enhanced, at least post-natally, by the Tph2 deletion (FIG. 1E-F). Conversely, blood serotonin levels were normal in Tph2−/− mice (FIG. 1G). Thus, the Tph2−/− mouse is an animal model lacking serotonin selectively in the brain. Serum levels of leptin, insulin, corticosterone, and T4, as well as body length, were normal in Tph2−/− animals (FIG. 1H and FIG. 8E).

### Brain-Derived Serotonin Increases Appetite and Energy Expenditure

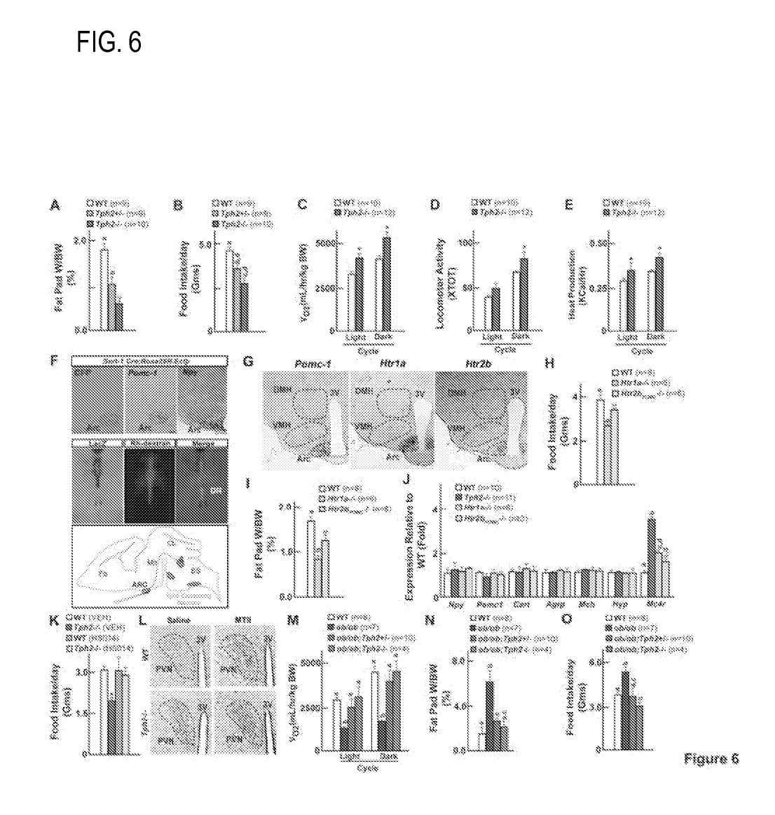

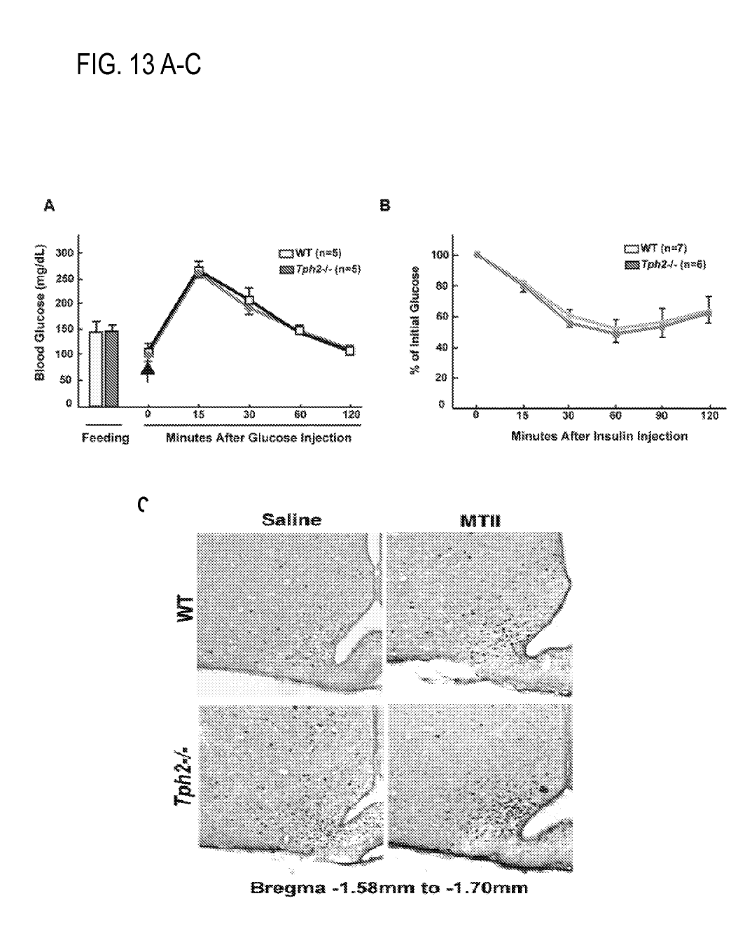

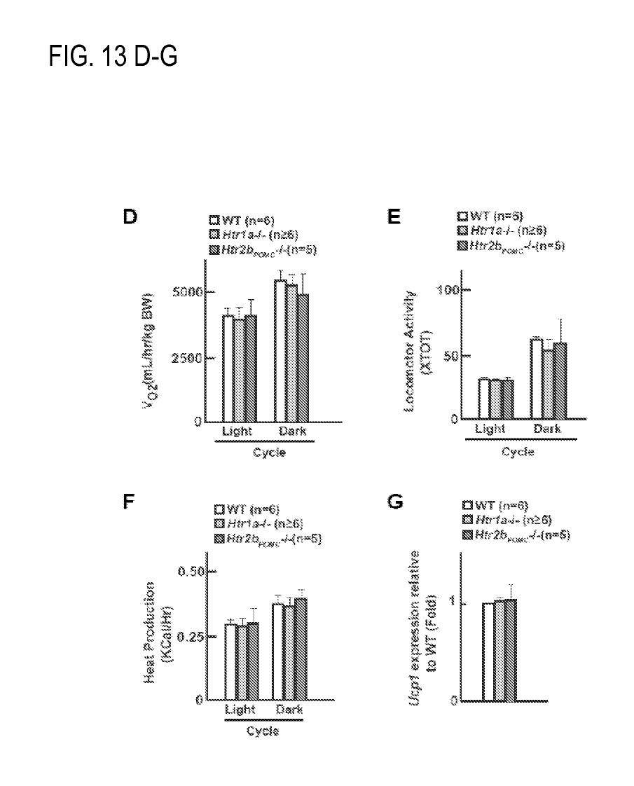

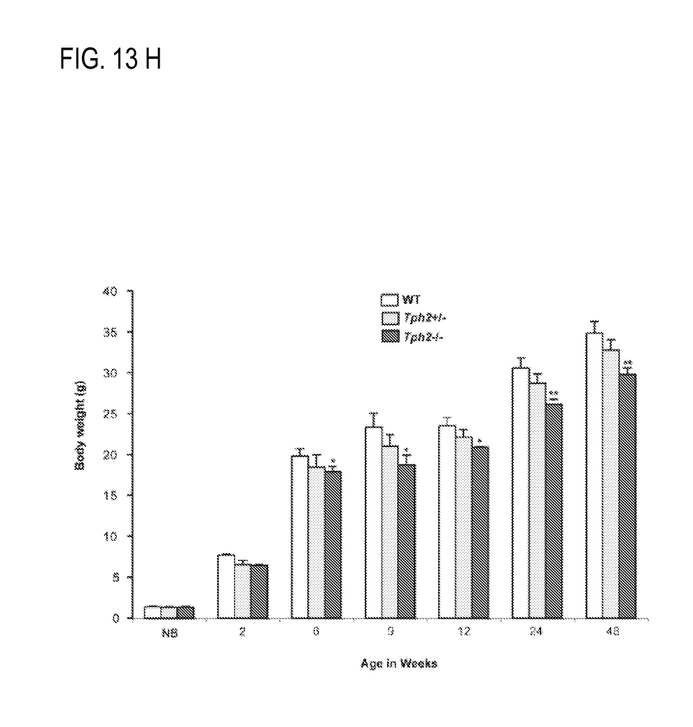

A significant decrease in fat pad weight in Tph2−/− mice was consistently observed (FIG. 6A). This surprising observation led to the analysis in greater detail of energy metabolism in these mutant mice. At both 6 and 12 weeks of age, there was a significant decrease in food intake in Tph2−/− (−31%) and Tph2+/− (˜14%) mice compared to WT littermates, along with an increase in energy expenditure (as measured by VO₂, XTOT and heat production) (FIG. 6B-E). In contrast, glucose metabolism as well as serum levels of leptin and other hormones were not affected in Tph2-deficient mice (FIG. 1H, FIG. 8E, and FIG. 13A-B). These results showed that BDS increases appetite and reduces energy expenditure.

### Leptin Regulates Appetite and Energy Expenditure Via Signaling in Serotonergic Neurons

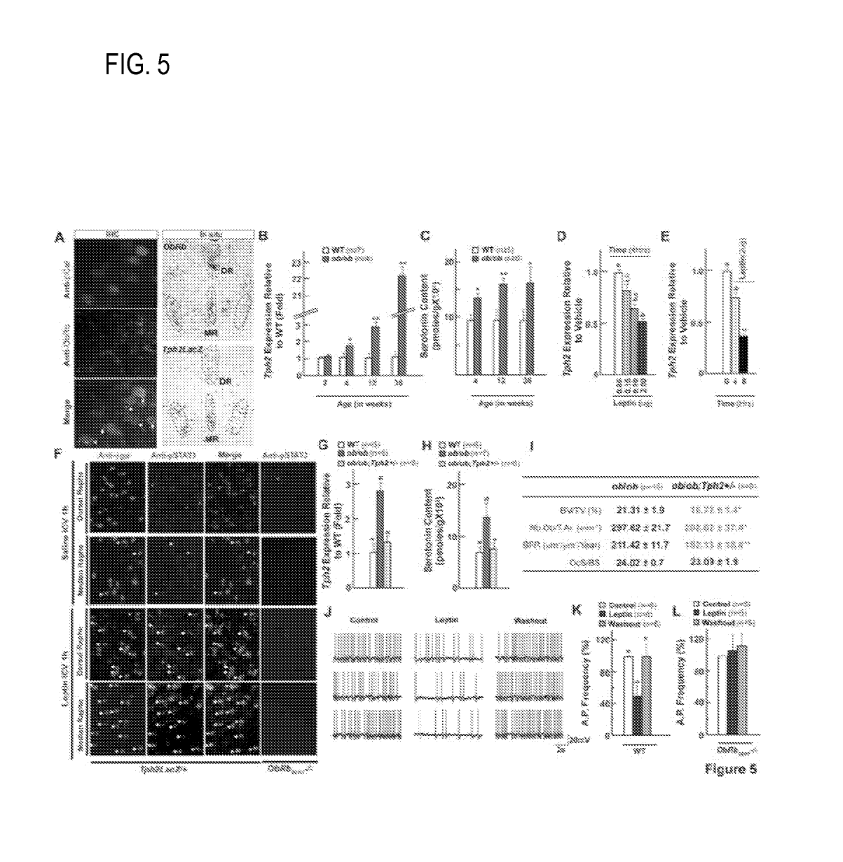

Obese mice that have a haploid complement of Tph (Ob/ob; Tph2+/− mice) have normal brain serotonin levels (FIG. 5H). Remarkably, ob/ob; Tph2+/− mice also had appetite and energy expenditure parameters that were indistinguishable from WT littermates (FIG. 6M-O and data not shown), suggesting that leptin inhibits BDS synthesis in order to decrease appetite and to increase energy expenditure. Consistent with this hypothesis, ob/ob mice in which the Tph2 gene was knocked out (ob/ob; Tph2−/−) were unable to synthesize serotonin at all in the brain and they had an even a lower appetite than WT mice; as a result, their fat pad weights were significantly smaller than the fat pad weights of ob/ob littermates with normal Tph (FIG. 6M-O).

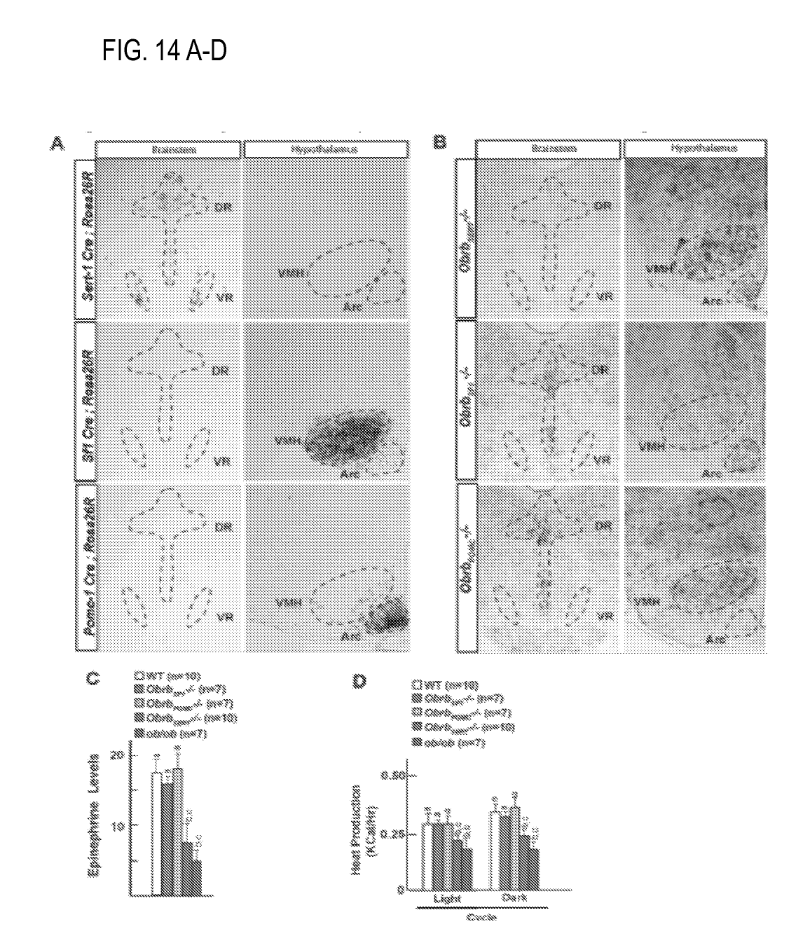

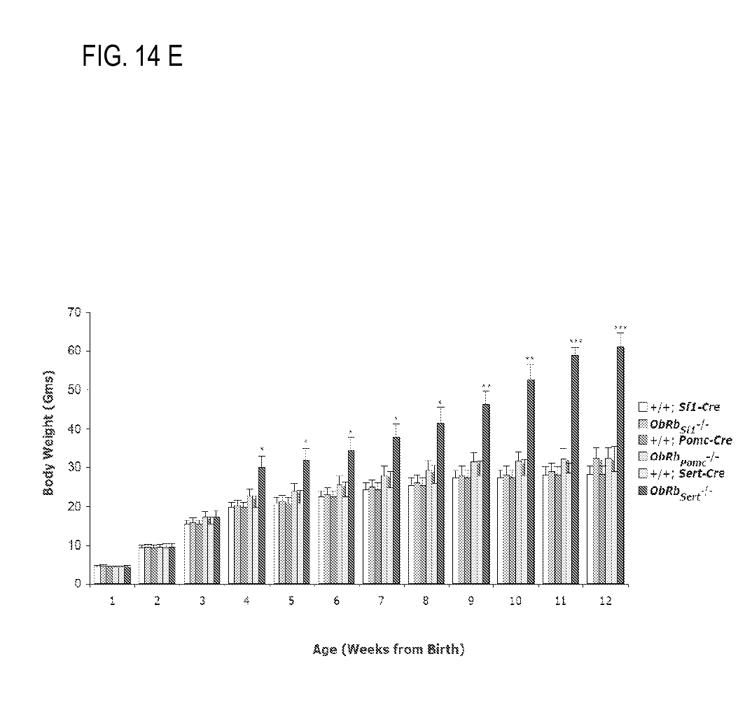

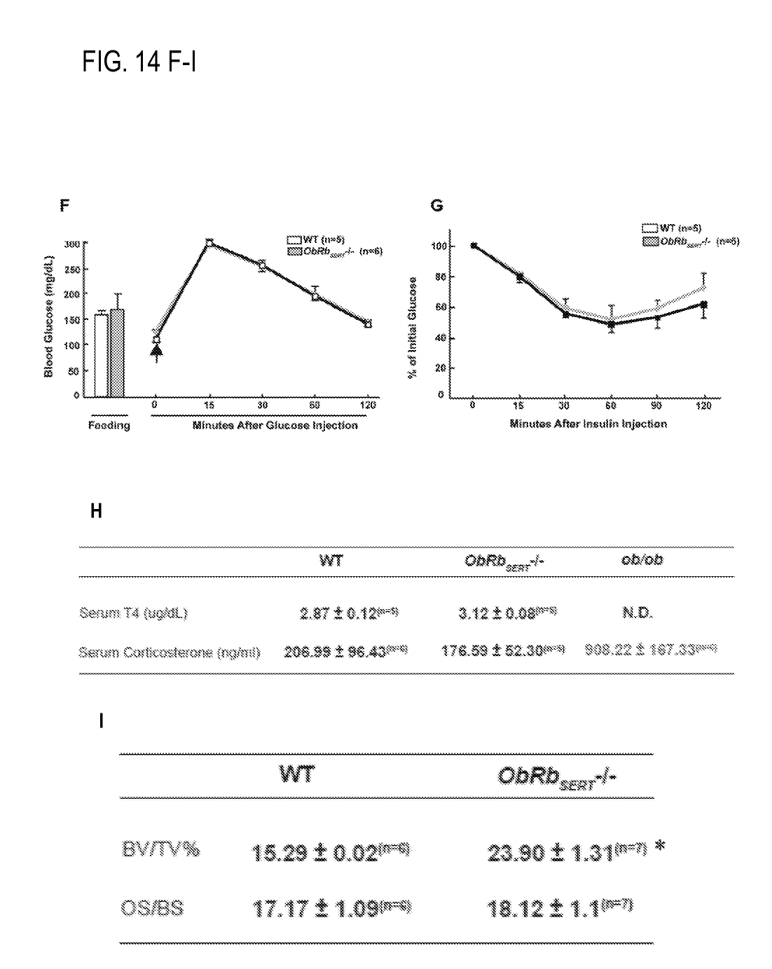

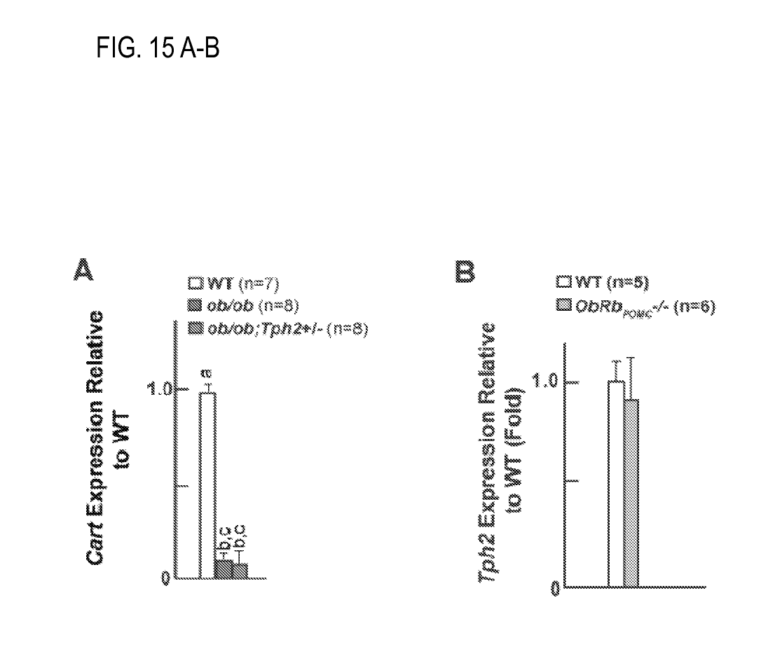

Bone mass, appetite and energy expenditure in mouse strains lacking leptin receptors (ObRb) in distinct neuronal populations in the brain were studied to establish that serotonergic neurons of the brainstem and BDS are a critically important entry point and target of leptin in the brain, (FIG. 14A-B) were analyzed. This analysis was performed on mice fed a normal diet since leptin signaling-deficient mice develop a massive obesity on this diet. The specificity of Cre expression was verified for each mouse line by crossing it with RosaR26 mice and by in situ hybridization (Soriano, 1999, Nat. Genet. 14:670-689) (FIG. 14A-B). The arcuate nucleus (or infundibular nucleus) is an aggregation of neurons in the mediobasal hypothalamus, adjacent to the third ventricle and the median eminence. The ventromedial nucleus (sometimes referred to as the ventromedial hypothalamus) is a nucleus of the hypothalamus that is most commonly associated with satiety.

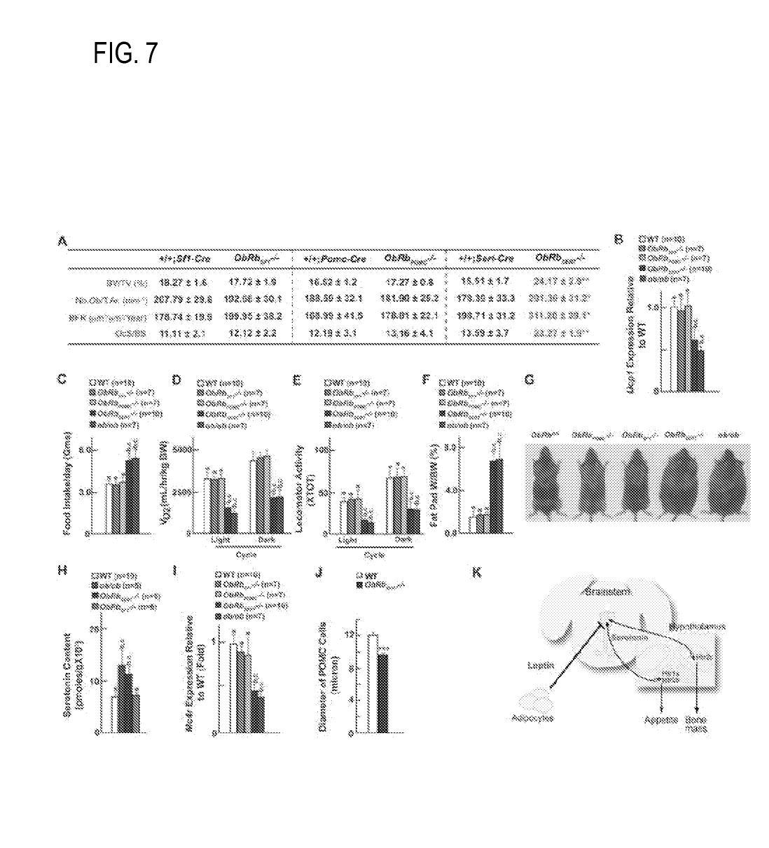

As reported previously, mice lacking ObRb selectively either in Sf1-expressing neurons of the ventromedial hypothalamus (VMH) nuclei or in Pomc-expressing neurons of the arcuate nuclei had normal sympathetic activity, bone remodeling parameters and bone mass; they also had normal appetite, energy expenditure and body weight when fed a normal diet (FIG. 7A-G and FIG. 14A-I) (Balthasar et al., 2004, Neuron 42:983-991; Dhillon et al., 2006, Neuron 49.191-203). These results show that leptin does not act in the hypothalamus or arcuate nuclei to control these parameters. In contrast, ObRb_{SERT}−/− mice lacking ObRb selectively in serotonergic neurons of the brainstem rapidly developed a low sympathetic activity, high bone mass phenotype, and an increase in appetite similar to that of ob/ob mice; they also had low energy expenditure (FIG. 7A-G). These ObRb_{SERT}−/− mice developed an obesity phenotype of similar severity and at a similar pace to mice lacking leptin signaling when fed a normal diet (FIG. 7G and FIG. 14E). Serotonin in the brain of ObRb_{SERT}−/− was elevated to the same extent as in ob/ob mice, while it was normal in the brain of ObRb_{SF1}−/− mice (FIG. 7H) Remarkably, hypothalamus gene expression analysis by real-time PCR revealed a decrease in Mc4r and Pomc expression, and an increase in Npy and Agrp expression in ObRb_{SERT}−/− mice that is of similar severity to the one observed in ob/ob mice (FIG. 7I).

Cell-specific gene deletion of the leptin receptor shows that leptin inhibits the effect of serotonin on appetite and increases energy expenditure because it reduces serotonin synthesis in the brainstem and reduces the firing of serotonergic neurons (FIG. 7K). Accordingly, while abrogating BDS synthesis corrects the increased appetite and decrease in energy expenditure phenotypes caused by leptin deficiency, inactivation of the leptin receptor in serotonergic neurons recapitulates those phenotypes fully.

It was observed that ObRb deletion in Tph2-expressing neurons in the brainstem also had an organizational effect on Pomc-expressing neurons of the arcuate nuclei. Indeed, the average diameter of Pomc-expressing neurons in Tph2-expression, ObRb_{SERT}−/− mice (n=42) was significantly lower than in WT mice (FIG. 7J). It has been shown that ob/ob mice also have a lower POMC perikaryal diameter that is associated with a ˜50% decrease in perikaryal synapse density of POMC neurons (Pinto et al., 2004, Science 304:110-115). Altered synaptic input organization of POMC neurons was also detected in ObRb_{SERT}−/− mice (14.76±1.3 vs 27.31±2.03 synapses per 100 micron perikaryal membrane in ObRb_{SERT}−/− and WT mice respectively). Thus, it is likely that the ob/ob phenotype of POMC neurons is determined, at least in part, by leptin signaling in serotonergic neurons of the brainstem.

A new tamoxifen-inducible Tph2-Cre transgenic mouse model was developed to permit the selective deletion of a target gene only in serotonergic neurons to facilitate experiments whether control of appetite in mice fed a normal chow is regulated by leptin signals in brainstem neurons. Cre cDNA was inserted at the ATG of the Tph2 gene in a BAC clone containing the entire mouse Tph2 gene. This construct should drive the expression of the Cre recombinase under the control of Tph2 regulating elements. To ascertain the cell-specific activity of the regulatory elements contained in the bacterial artificial chromosome (BAC), Tph2-Cre transgenic mice were crossed with Rosa26R mice (Soriano 1999, Nat Genet. 14:670-689) In Rosa26R mice the β-Galactosidase reporter gene containing a floxed transcriptional blocker cassette inserted between the transcription start site and the ATG is placed downstream of the Rosa26 promoter. Thus, β-Galactosidase can only be expressed after Cre-mediated deletion of the transcriptional blocking cassette. Following treatment of 6-week-old mice with tamoxifen (1 mg/20 g body weights during 5 days successively) (analysis done 10th July) β-Galactosidase staining showed that this construct drives gene expression in serotonergic neurons of the brainstem (stained in blue) but not in any other part of the brain, including the hypothalamic. In this model any phenotype seen in mutant mice generated using Tph2-cre transgene to delete a gene of interest could only occur by the expression of the transgene in serotonergic neurons.

Using the exquisite specificity of the Tph2-Cre transgene leptin receptors (Obrb) were deleted in serotonergic neurons of brainstem specifically after birth. Tamoxifen (1 mg/20 g body weight) was injected every day for 5 days intra-peritoneal injection in 6 week-old WT and Tph2-Cre; Obrb^{f/f} mice with daily weighing. Tph2-Cr; Obrb^{f/f} mice gained significantly more weight than WT mice, and appetite was significantly increased 6 weeks after the end of this tamoxifen treatment, while energy expenditure significantly decreased. Taken together these results indicate that the absence of leptin signaling in serotonergic neurons of the brainstem results in hyperphagia and decrease in energy expenditure that is similar to what is observed in leptin-deficient ObOb CONFIRM mice during adulthood.

Leptin Inhibits the Neuronal Activity of Serotonergic Neurons

The mediation of peripheral hormone action on the output of the brain relies on altered neuronal circuit activity. Interaction between neuronal circuits hinges on electric properties of neurons, particularly on the generation of action potentials. Thus, to test whether leptin directly alters serotonin output from brainstem neurons, the responses of serotonin-producing cells to leptin were analyzed with whole cell patch clamp recording in brain slices containing dorsal raphe (DR). Slices were taken from WT animals and from mice lacking ObRb selectively in Tph2-expressing neurons (ObRb_{SERT}−/− mice). Serotonergic neurons were identified according to their unique properties (long-duration action potential, activation by norepinephrine and inhibition by serotonin itself) (Liu et al., 2002, J. Neurosci. 22:9453-9464). Since serotonergic neurons are usually quiescent in slices because of the loss of noradrenergic inputs, action potentials in these neurons were restored by application of alpha-1 adrenergic agonist phenylephrine (3 μM) in the bath (Liu et al., 2002, J. Neurosci. 22:9453-9464).

Whole cell patch recording showed that leptin significantly decreased action potential frequency in serotonergic neurons of WT mice, but not in serotonergic neurons of mice lacking ObRb in Tph2-expressing neurons (ObRb_{SERT}−/− mice). Thus leptin can directly alter the activity of serotonergic neurons in the brainstem and that this effect is mediated by leptin receptors (ObRb) expressed on these neurons.

### Serotonergic Neurons in the Brainstem Project to the Arcuate Nuclei in the Hypothalamus

Multiple lines of evidence have established that neurons of the arcuate nuclei of the hypothalamus are implicated in the regulation of appetite and energy expenditure (Cohen et al., 2001; Cowley et al., 2001; Heisler et al., 2003). Hence, we asked whether it is through its expression in these neurons that the Htr1a receptor regulates appetite. The observations relating to appetite and energy expenditure in the Tph2−/− knockout mice, along with the fact that the control of appetite and energy expenditure requires the integrity of the hypothalamus raised the prospect that axonal projections emanating from Tph2-expressing neurons in the brainstem reach arcuate nuclei to regulate these functions. Hetherington and Hanson; Hypothalamic lesions and adiposity in rats. Anat Rec, 78:149-172, 1940

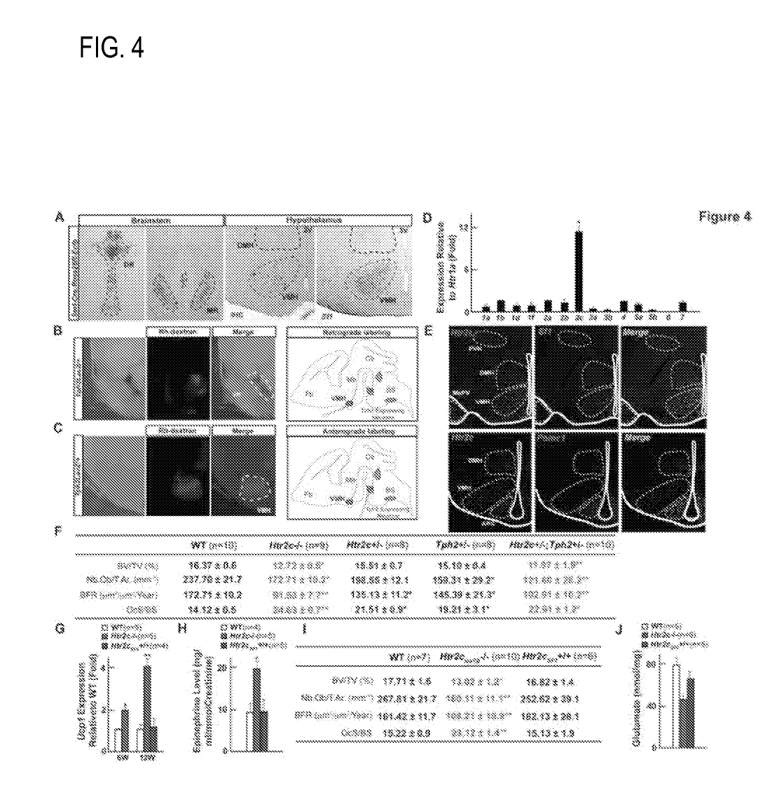

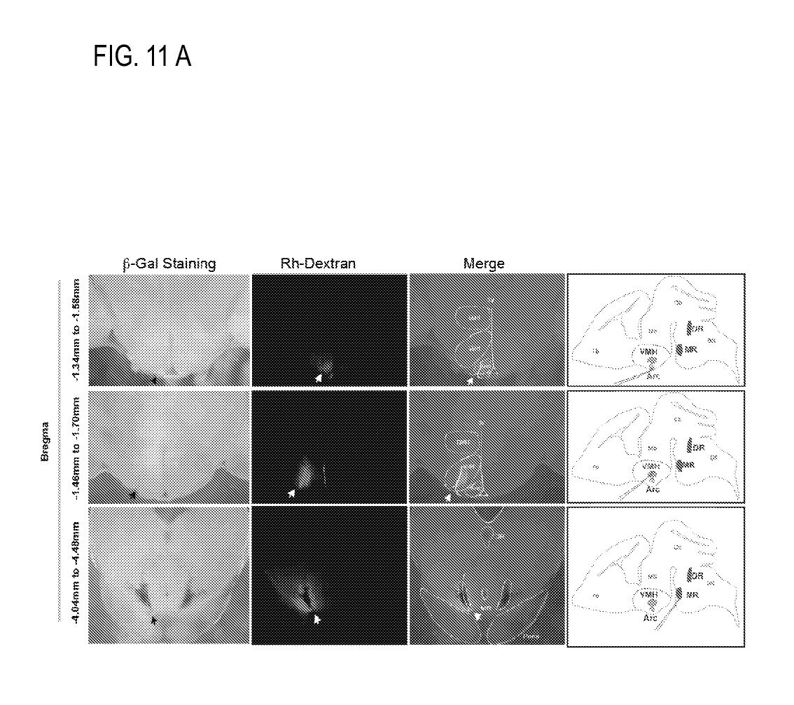

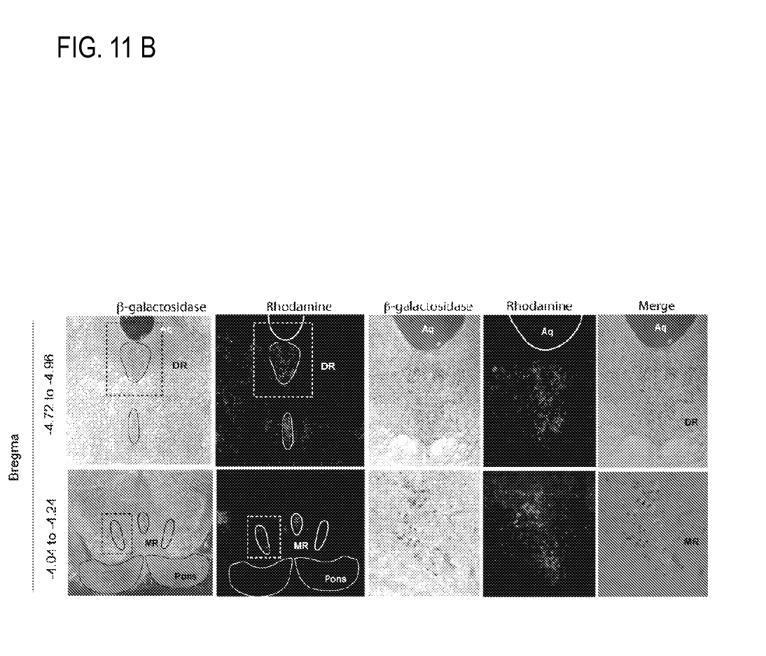

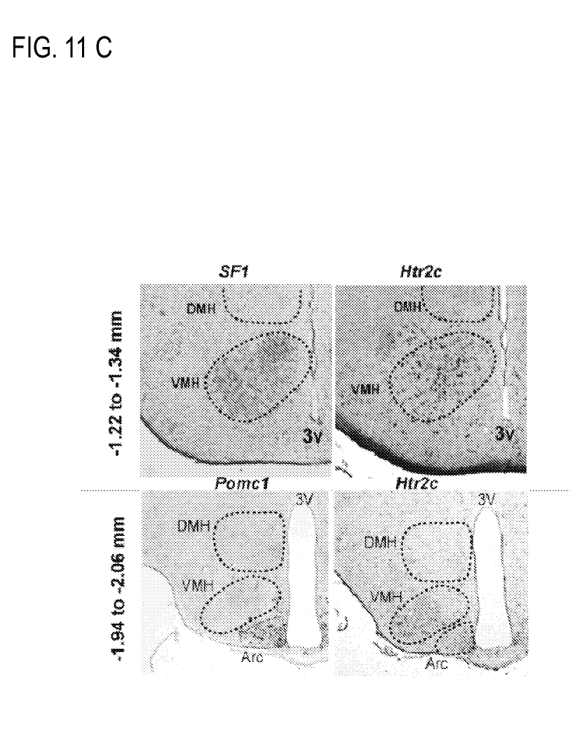

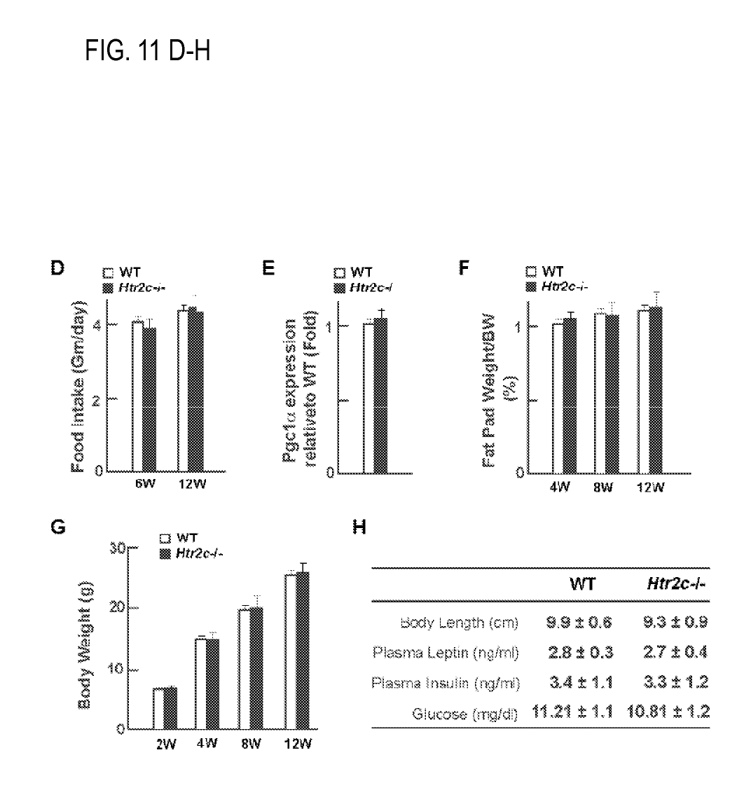

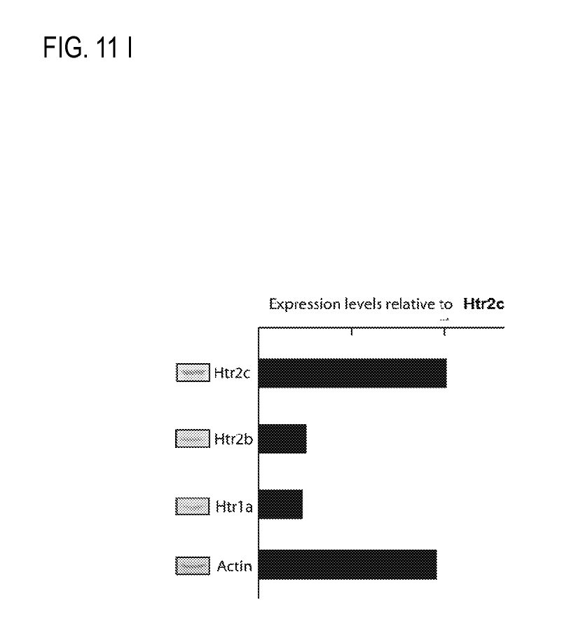

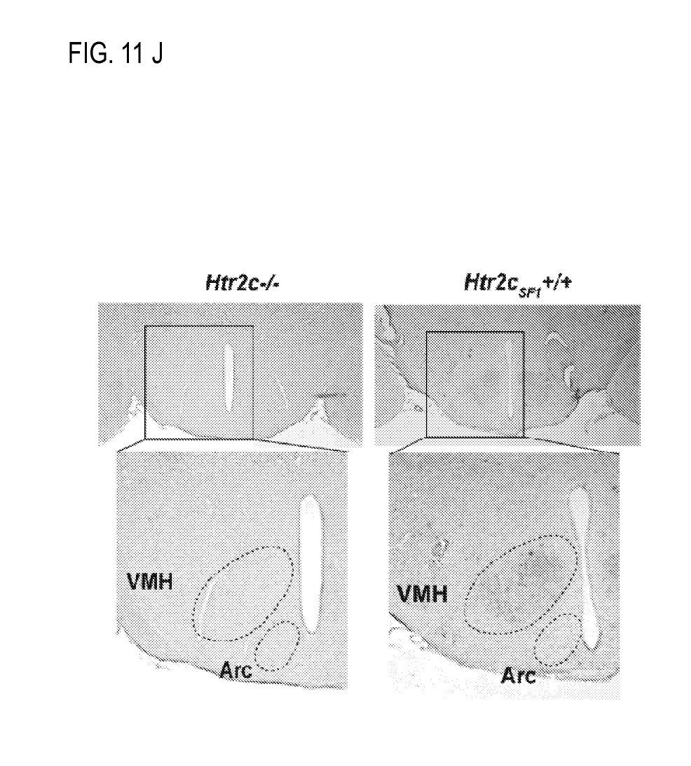

To search for anatomical connections between Tph2-expressing and hypothalamic neurons Rosa26R-Ecfp mice were used (Srinivas et al., 2001, BMC Dev. Biol. 1:4). In this mouse model, the Ecfp (enhanced cyan fluorescent protein) reporter gene containing a floxed transcriptional blocker cassette inserted between the transcription start site and the ATG translation initiation site is placed downstream of the Rosa26 promoter. Thus, Ecfp can only be expressed after Cre-mediated deletion of the transcriptional blocker. Rosa26R-Ecfp mice were crossed with Sert-Cre transgenic mice that express Cre only in Tph2-expressing neurons of the brainstem (Zhuang et al., 2005, J. Neurosci. Methods 143:27-32). Ecfp immunostaining in Sert-Cre/Rosa26R-Ecfp mice showed that axons emanating from Tph2-expressing neurons of the brainstem projected to the hypothalamus (FIG. 4A) and in situ hybridization performed on adjacent sections demonstrated that those axonal projections reached Sf1-expressing VMH neurons (FIG. 4A). These findings were confirmed by fluorescent dextran tracing. Anterograde and retrograde labelling in Tph2+/− mice showed that VMH neurons were targeted by neuronal projections emanating from Tph2-expressing neurons in the brainstem (FIG. 4B-C and FIG. 11A-B). These morphological data suggest that serotonin signals in neurons of the VMH nuclei. Further studies showed that the expression of Pomc-1 and Npy, two arcuate neuron-specific genes, were analyzed on adjacent sections in Sert-Cre/Rosa26R-Ecfp mice (FIG. 6F and FIG. 11B). This analysis verified that neurons of the arcuate nuclei were targeted by serotonergic innervations emanating from the brainstem, an observation confirmed in the Tph2+/− mice by retrograde labeling of the projections reaching the serotonergic neurons of the brainstem (FIG. 6F).

Serotonergic Neurons in the Brainstem Projecting to the Arcuate Nuclei in the Hypothalamus Regulate Appetite and Energy through Htr1a and Htr2b Receptors

Real-time PCR analysis of Tph−/− mutants revealed that, among the 14 serotonergic receptors, Htr2c was by far the most highly expressed in the hypothalamus, albeit it was not the only one (FIG. 4D). Double fluorescent in situ hybridization experiments showed that Htr2c was expressed in Sf1-expressing ventromedial hypothalamus (VMH) and, to a lower extent, in Pomc-expressing arcuate neurons (Pasqualetti et al., 1998, Ann. NY Acad. Sci. 861:245) (FIG. 4E and FIG. 11C)).

Among all serotonin receptors, the most highly expressed in arcuate neurons in the hypothalamus of normal mice were Htr1a, and, to a lower extent Htr2b and Htr2c (FIG. 6G and FIG. 11C). While food intake was not affected in Htr2c−/− mice, it was significantly reduced in mice lacking Htr1a in all cells (Htr1a knockouts: Htr1a−/−) (˜24% reduction), or lacking Htr2b in arcuate neurons only (Htr2bPOMC−/− mice) (˜10% reduction). Fat pad weight was also lower in Htr1a−/− and Htr2bPOMC−/− mice (FIG. 6H-I and FIG. 11D).

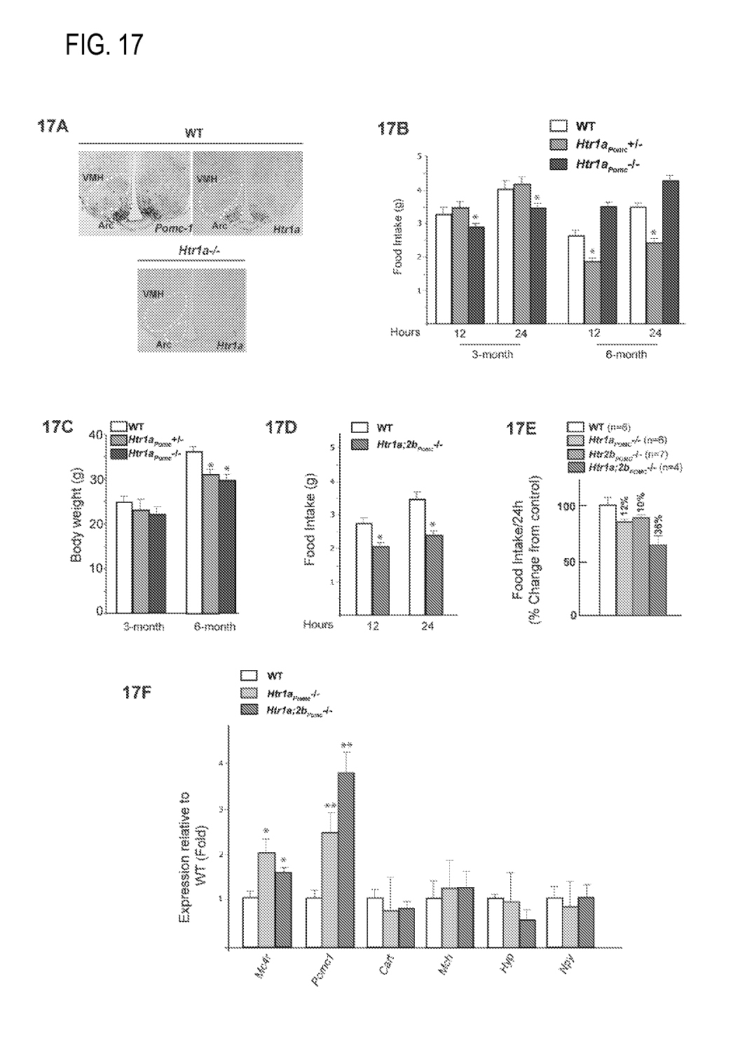

In additional studies the Htr1a receptor was conditionally inactivated by crossing mice harboring a floxed allele of Htr1a with Pomc-Cre transgenic mice that express Cre only in Pomc-expressing neurons of the arcuate nuclei (Balthasar et al., 2004). In situ hybridization analysis ascertained that Htr1a expression in the arcuate neurons was completely ablated in Htr1a_{Pomc}−/− mice (FIG. 17A). As can be seen in FIG. 17B, 3-month-old Htr1a_{Pomc}−/− mice demonstrated a significant reduction in their food intake although it was milder than in mice lacking this receptor in all cells (Yadav et al., 2009). As expected this decrease in food intake was associated with a significant decrease in the body weight of Htr1a_{Pomc}−/− mice (FIG. 17C). As Htr2b, another serotonin receptor affecting appetite, is also expressed in Pomc-expressing neurons we next generated mutant mice lacking both Htr1a and Htr2b in Pomc-expressing neurons (Htr1a; 2 b_{Pomc}−/− mice). Appetite in these double mutant mice was significantly lower than a more additive effect of the two mutations would have predicted (FIG. 17 D-E). To demonstrate molecularly that the deletion of Htr1a and of Htr1a and Htr2b from Pomc-expressing neurons could affect appetite we measured expression of genes, such as Pomc-1 and Mc4r that contribute to the regulation of appetite, whose expression in the hypothalamus is decreased by the absence of leptin or leptin signaling in serotonergic neurons. As shown in FIG. 17F and consistent with their decreased appetite, expression of these two genes was increase in the hypothalami of Htr1a_{Pomc}−/− and Htr1a; 2 b_{Pomc}−/−. Taken together, these results establish that serotonin signals in Pomc-expressing neurons through Htr1a and Htr2b has a synergistic effect on appetite.

BDS Regulation of Appetite Occurs through the Htr1a and Htr2b Receptors and Involves Melanocortin and CREB Signaling in the Hypothalamus

A survey was carried out of the expression of genes in hypothalamic neurons that may mediate leptin regulation of appetite and the expression of which is perturbed in Tph2−/− mice. Among those tested, the only gene whose expression was significantly increased in Tph2−/− mice was Mc4r (FIG. 6J), a gene the inactivation of which in mice and humans causes hyperphagia and obesity (Huszar et al., 1997, Cell 88:131-141; Yeo et al., 1998, Nat. Genet. 20:111-112). Two observations support the notion that the appetite phenotype of the Tph2−/− mice is caused, at least in part, by an increase in melanocortin signaling. First, ICV infusion of an Mc4r antagonist (HS014) increased appetite ˜50% in Tph2−/− mice (FIG. 6K); second, ICV infusion of a Mc4r agonist (MTII) increased c-Fos expression in neurons of the paraventricular and arcuate nuclei of both WT and Tph2−/− mice (FIG. 6L and FIG. 13C). Moreover, Mc4r expression was increased ˜2 fold in Htr1a−/− and ˜1.6 fold in Htr2b_{Pomc}−/− mice, but was unaffected in Htr2c−/− mice (FIG. 6J and data not shown). Energy expenditure was normal in Htr1a−/− and Htr2b_{Pomc}−/− mice, indicating that serotonin uses other receptors, yet to be identified, to regulate this function (FIG. 13D-G). Taken together, these results indicate that BDS regulates appetite and energy expenditure and that for the control of appetite this mediation occurs through the Htr1a and Htr2b receptors and involves melanocortin signaling.

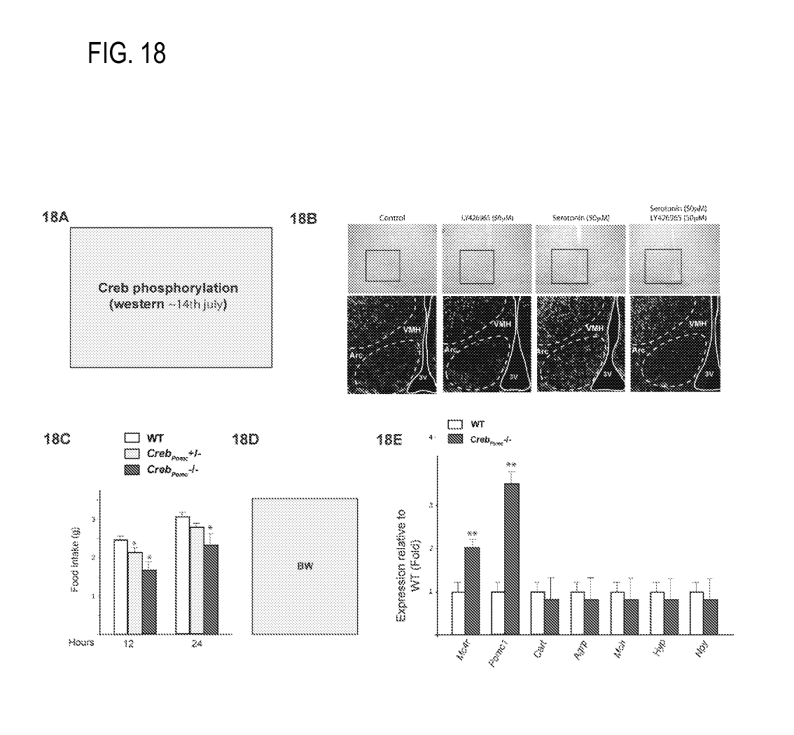

Htr1a is a Gs-protein coupled receptor that signals through the cAMP-PKA-dependent pathway. The main transcription factor downstream of this pathway is CREB which has been shown already to mediate two other homeostatic functions of serotonin (Yadav et al., 2008; “Oury et al., 2010”). The following experiments show that CREB is also involved in the serotonin regulation of appetite through its expression in neurons of the arcuate nuclei. Immunofluorescence of p-CREB from hypothalamic explants cultures showed that serotonin treatment of explants increased CREB phosphorylation in arcuate neurons (FIG. 18 A-B). To establish that in vivo CREB, through its expression in arcuate neurons, mediates serotonin regulation of appetite, mice lacking this gene in Pomc-expressing neurons (Creb_{Pomc}−/− mice) were generated. Creb_{Pomc}−/− mice showed a significant reduction in food intake and a reduced body weight demonstrating that CREB signaling in the Pomc-expressing neurons regulates food intake (FIG. 18 C-D). Furthermore, the expression of genes inhibiting food intake such as Mc4r and Pomc-1 was significantly increased in Creb_{Pomc}−/− hypothalami (FIG. 18 E). Based on these observations certain embodiments of the invention are directed to methods to reduce appetite and increase energy expenditure by administering a therapeutically effective amount of a CREB antagonist to a patient in need of such treatment. CREB antagonists include: ICER (Jaworski et al. 2003 Journal of Neuroscience) and CREB-M1 (Dworkin et al., 2007 Developmental biology).

To determine that this function of CREB occurs, at least in part, following serotonin signaling in these neurons compound heterozygous mice lacking one copy of CREB and one copy of Htr1a in Pomc-expressing neurons of the arcuate nuclei were generated. As shown in FIG. 18F these mice showed a decrease in appetite confirming this hypothesis that CREB is a transcriptional effector of serotonin regulation of appetite.

### Pharmacological Targeting of Htr1a Receptors Decreases Appetite in Mice

More evidence confirming that serotonin increases appetite through the Htr1a receptor and leptin inhibits this action would show that inhibition of serotonin signaling through the Htr1a receptor decreases appetite in Wt mice, and leptin inhibits appetite by decreasing serotonin synthesis and release. In this case leptin should decrease appetite in ob/ob mice that are leptin-deficient. This last point is needed to validate the notion that leptin decreases appetite by inhibiting sympathetic tone in the brainstem.

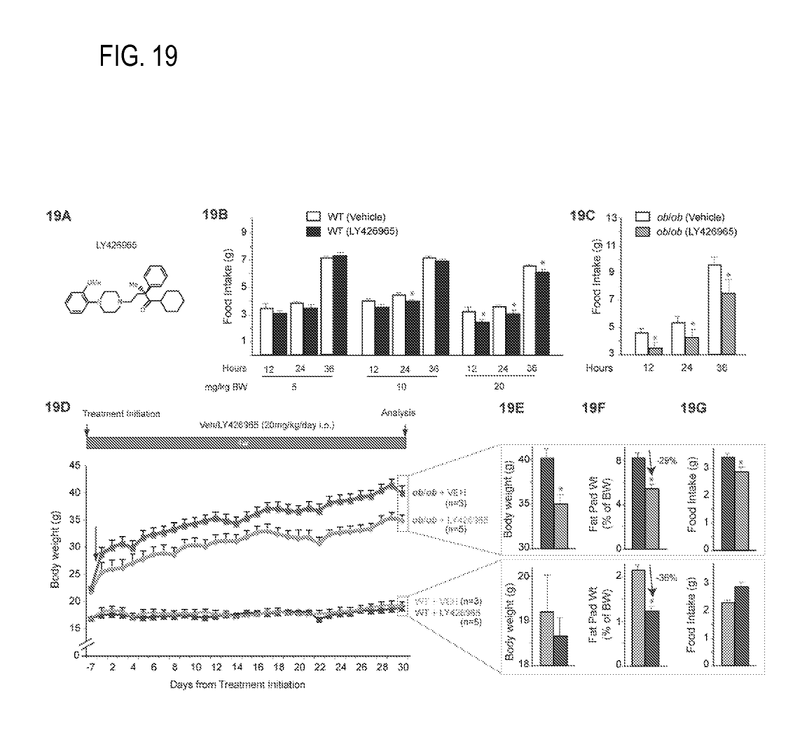

These two hypotheses were tested through the use of a small molecule that selectively antagonizes signaling through Htr1a receptor. Other Htr1a and 2b receptor antagonists for use in the present invention are discussed below. This molecule (LY426965) has high affinity for the Htr1a receptor (Ki=4.66 nM) and 20-fold or greater selectivity over other serotonin and non-serotonin receptor subtypes (Rasmussen et al., 2000). That appetite was not decreased in Htr1a_{Pomc}−/− mice treated with LY426965 strongly suggest that this compound acts only as an inhibitor of Htr1a signaling (FIG. 19 A) and does not affect Htr2b receptor activity.

Doses of LY426965 ranging from 1 to 20 mg/kg of body weight fed to 12 week-old C57B16/J mice caused a dose-dependent decrease in appetite (FIG. 19 B). This decrease in food intake reached 77% of control values when using 20 mg/kg of the compound (FIG. 19 C). Mass spectrometric analysis of mice hypothalami after administration of the compound verified that LY426965 could reach the hypothalamus (FIG. 19 D). Thus LY426965 decreases appetite in WT mice by inhibiting serotonin signaling through the Htr1a receptor that is located in the hypothalamus.

To determine whether LY426965 could decrease appetite in leptin-deficient ob/ob mice, we administered 4-week old ob/ob mice with a single dose of LY426965 (0.2 mg/20 g of body weight) and measured food intake 12, 24 and 36 h later. Food intake in LY426965-treated animals was 20-25% lower than in vehicle-treated mice at all time points analyzed demonstrating that inhibition of signaling through Htr1a in ob/ob mice can reduce appetite FIG. 19 D That this compound did not rescue fully the obese appetite phenotype in leptin-deficient mice is consistent with the notion that serotonin decreases appetite by signaling also through another receptor, the Htr2b receptor. Experiments were conducted to determine whether LY426965 could rescue, at least in part, the obesity of ob/ob mice if administered chronically, once mice had already developed an obesity phenotype. 4-week old ob/ob mice were administered daily with 20 mg/kg body weight dose of LY426965 for 4 additional weeks. The results presented in FIG. 19 E-G show that LY426965-mediated suppression of Htr1a receptor signaling significantly decreased the obesity phenotype of adult ob/ob mice. This result is consistent with the notion that one mechanism whereby leptin inhibits appetite is by decreasing serotonin synthesis and release from brainstem neurons (Yadav et al., 2009).

These experiments support embodiments of the invention using Htr1a antagonists to treat eating disorders by decreasing appetite and using agonists of Htr1a to increase appetite.

### Brain-Derived Serotonin Regulation of Bone Mass

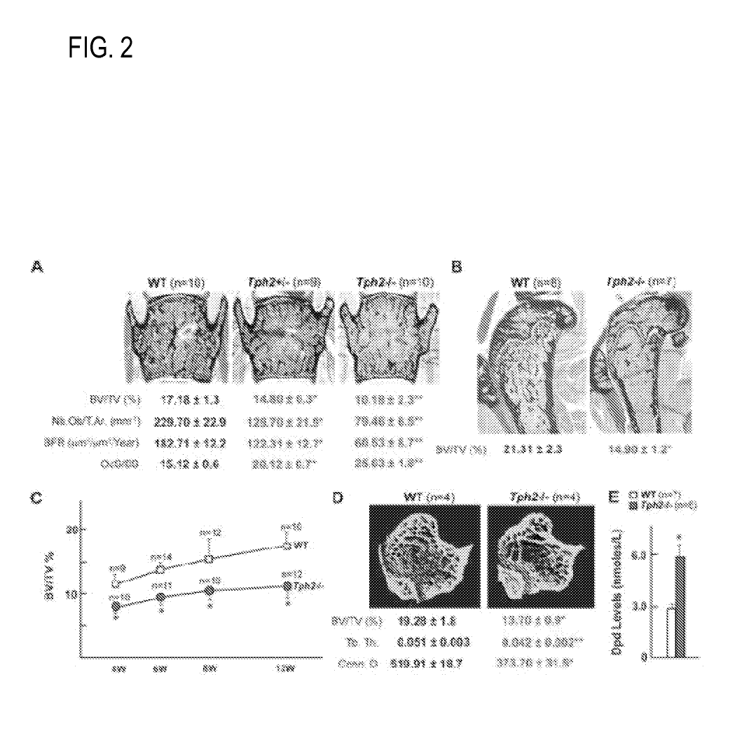

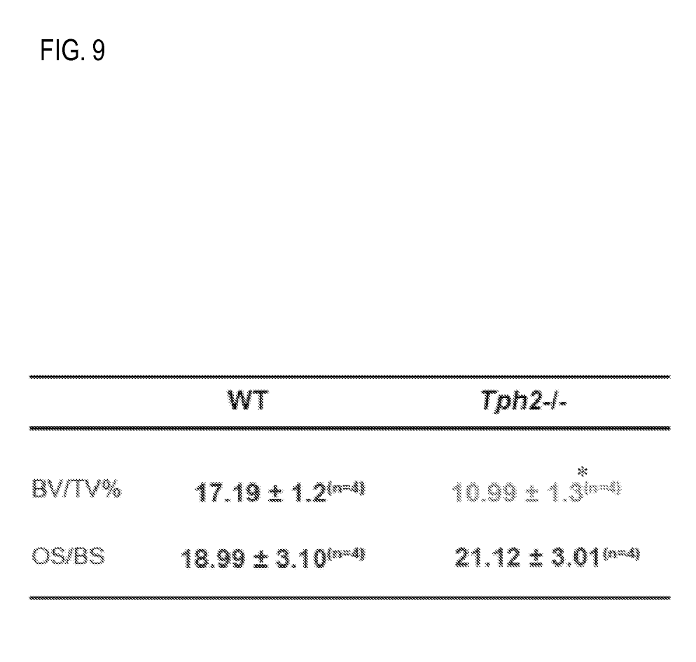

One mediator linking leptin signaling in the brain to bone remodeling is the sympathetic tone, which inhibits bone formation and favors bone resorption through the β2 adrenergic receptor (Adrβ2) expressed in osteoblasts (Elefteriou et al., 2005, Nature 434:514-520; Takeda et al., 2002, Cell 111:305-317). Hence, sympathetic activity can be used as a readout of leptin regulation of bone mass. To assess the influence of BDS on bone remodeling, histological, histomorphometric and microcomputed tomography (μCT) analyses of bones were performed in 4, 6 and 12 week-old wild type (WT) and BDS knockout Tph2−/− mice. The absence of serotonin in the brain resulted, at all time points, in a severe low bone mass phenotype affecting the axial (vertebrae) and appendicular (long bones) skeleton while bone length and width were unaffected (FIG. 2A-D and data not shown). Three month-old Tph2+/− mice also displayed a decrease in bone mass, albeit milder (FIG. 2A). This phenotype was secondary to a decrease in bone formation parameters (osteoblast numbers and bone formation rate) and to an increase in bone resorption parameters (osteoclast surface and circulating levels of deoxypridinoline (Dpd), a degradation product of type I collagen and a biomarker of bone resorption (Eyre et al., 1988, Biochem 252:494-500)) (FIGS. 2A and E). Bone mineralization was normal in Tph2−/− mice (FIG. 9). These results demonstrate that BDS is a positive and powerful regulator of bone mass accrual, acting on both arms of bone remodeling. Since serotonin does not cross the blood brain barrier, these observations provide a rare example of the regulation of bone mass by a neuromediator. The influence of brain-derived serotonin on increasing bone mass prevails over the influence of gut-derived serotonin which increases bone mass. (International Patent Publication WO 2009/045900).

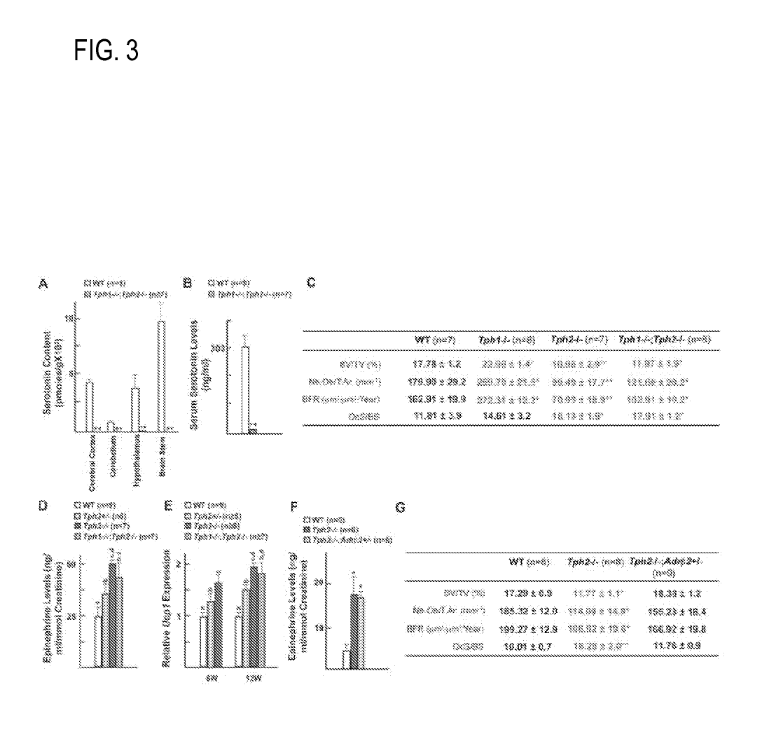

That serotonin exerts opposite influences on bone remodeling depending on its site of synthesis was unexpected. Since BDS accounts for only 5% of total serotonin, the actual contribution of BDS to the overall regulation of bone mass accrual by serotonin was investigated. To that end, mice were generated that were unable to synthesize serotonin anywhere in their body by inactivating both Tph1 and Tph2 (FIG. 3A-B). Tph1−/−; Tph2−/− mice were born at the expected Mendelian ratio and had normal size and life span (data not shown). Surprisingly, like the Tph2−/− mice, Tph1−/−; Tph2−/− mice displayed a low bone mass secondary to a decrease in bone formation and to an increase in bone resorption parameters and affecting the axial and appendicular skeleton (FIG. 3C and data not shown). By showing that the influence of BDS on bone remodeling prevails over the one exerted by gut-derived serotonin, even though it accounts for only 5% of the total pool of serotonin, this experiment underscored the importance of BDS in the regulation of bone mass and was an incentive to elucidate the mode of action of BDS.

### Sympathetic Mediation of Brain-Derived Serotonin Regulation of Bone Mass

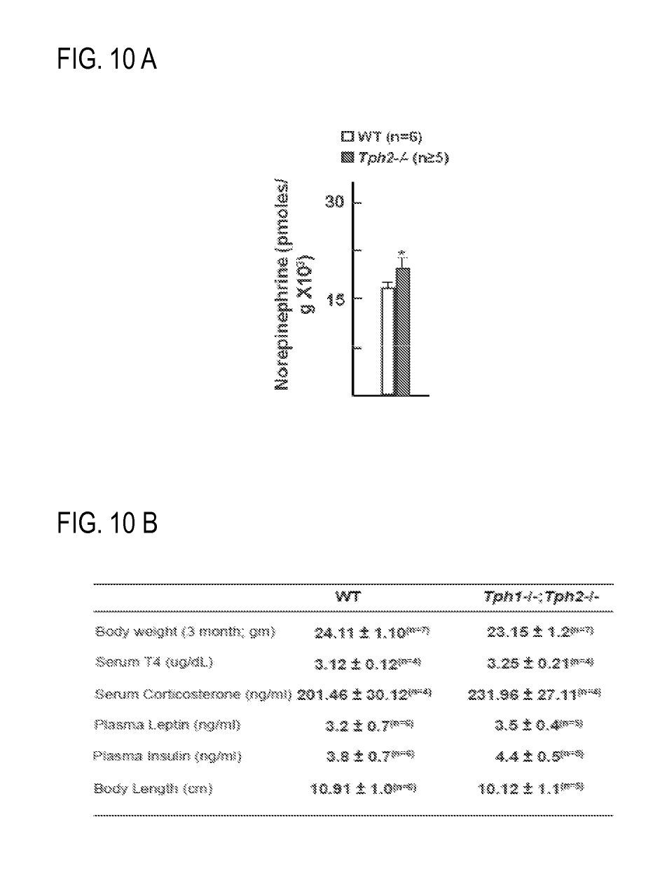

The decrease in bone formation and the increase in bone resorption seen in Tph2−/− mice is the mirror image of what is observed in mice lacking the β2 adrenergic receptor (Adrβ2−/− mice) (Elefteriou et al., 2005, Nature 434:514-520). This feature suggested that the bone phenotype of the mice lacking serotonin in the brain could be secondary to an increase in sympathetic signaling in osteoblasts. That norepinephrine content in the brain, epinephrine elimination in the urine and Ucp1 expression in brown fat, three markers of the sympathetic tone, were all markedly increased in Tph2+/−, Tph2−/− and Tph1−/−; Tph2−/− mice at 6 and 12 weeks of age supported this hypothesis (FIG. 3D-F and FIG. 10). Tph2−/− mice in which one allele of Adrβ2 had been inactivated (FIG. 3F-G) were also generated. One copy of this gene was removed because Adrβ2 is the only adrenergic receptor expressed in osteoblasts (Takeda et al., 2002, Cell 111:305-317). Tph2−/−; Adrβ2+/− mice had normal bone formation and bone resorption parameters and a normal bone mass. The same was true for Tph2−/−; Adrβ2−/− mice (FIG. 3G and data not shown). These results indicate that the regulation of bone mass accrual by BDS occurs by decreasing the sympathetic tone.

Brain-Derived Serotonin Regulates Bone Mass Through the Hypothalamus

To determine the importance of serotonin signaling through Htr2c in the regulation of bone mass, mice lacking Htr2c in all cells (Htr2c−/− mice) were first analyzed. Since Htr2c−/− mice develop an increase in food intake and adiposity beyond 14 week of age (Tecott et al., 1995, Nature 374:542-546), 6 and 12 week-old animals were analyzed after verifying that at those ages appetite, energy expenditure, body weight, fat pad weights and hormonal profiles were identical in Htr2c−/− and WT mice (FIG. 11D-H).

Since the sympathetic regulation of bone mass requires the integrity of the VMH neurons of the hypothalamus (Takeda et al., 2002, Cell 111:305-317), whether the BDS regulation of bone mass also occurs through a VMH relay was investigated.

Histological analyses uncovered in both 6 and 12 week-old Htr2c−/− mice a severe low bone mass phenotype secondary to a decrease in the number of osteoblasts and bone formation rate, and to an increase in the number of osteoclasts and bone resorption parameters (FIG. 4F and data not shown). Moreover, Ucp1 expression in brown fat and urinary elimination of epinephrine were both significantly higher in Htr2c−/− mice, revealing the existence of a high sympathetic activity (FIG. 4G-H). Thus, both in terms of bone remodeling parameters and sympathetic tone, Htr2c−/− mice are a phenocopy of Tph2−/− mice at time points when no metabolic abnormalities could be found.

It is possible to identify functional interplay between two proteins through the generation of compound mutant mouse strains. When protein A interacts with protein B in the control or realization of a given function, mice lacking the gene coding for A or the gene coding for B have very similar phenotypes. As a result, and even though mice heterozygous for either mutation are indistinguishable from wild-type littermates, compound heterozygous mutant mice lacking one allele of A and one allele of B display in most cases the same phenotype as the one observed in A−/− or B−/− mice.

To establish that it is by signaling through Htr2c that BDS regulates bone mass, compound mutant mice lacking one allele of Tph2 and one allele of Htr2c (Tph2+/−; Htr2c+/− mice) were generated. These mutant mice presented at 6 and 12 weeks of age the same low bone mass/high sympathetic activity phenotype as the Htr2c−/− and Tph2−/− mice (FIG. 4F and data not shown). These results support the notion that BDS utilizes the Htr2c receptor to regulate sympathetic tone and bone mass independently of the influence it exerts through this receptor on energy metabolism.

To determine whether it is through its expression in VMH neurons that Htr2c regulates bone mass, mutant mice harboring a loxP-flanked transcriptional blocking (loxTB) cassette inserted in the Htr2c gene (loxTB Htr2c mice) (Xu et al., 2008, Neuron 60:582-589) were used. In these mice, disruption of Htr2c transcription can be alleviated in a cell population of choice by expression of the Cre recombinase in that cell population. Htr2c re-expression was targeted to VMH neurons by crossing loxTB Htr2c mice with Sf1-Cre mice (FIG. 11J). Histological analyses showed that re-expression of Htr2c receptor in VMH neurons (Htr2c_{SF1}+/+ mice) rescued entirely the bone mass phenotype observed in the absence of Htr2c (FIG. 4G-I). Moreover, Ucp1 expression in brown fat and urinary elimination of epinephrine were also similar between WT and Htr2c_{SF1}+/+ mice and levels of glutamate, an inhibitor of sympathetic tone, that were suppressed in Htr2c−/− hypothalami were partially restored in Htr2c_{SF1}+/+ hypothalami (FIG. 4G-H and J). These findings echo previous observations indicating that serotonin attenuates activation of noradrenergic neurons in the locus coeruleus (Aston-Jones et al., 1991, J. Neurosci. 11:760-769). Taken together, the results presented indicate that BDS acts on VMH neurons, through Htr2c, to decrease sympathetic activity and thereby favor bone mass accrual.

### Leptin Inhibits Bone Mass Accrual by Decreasing Brain-Derived Serotonin Synthesis

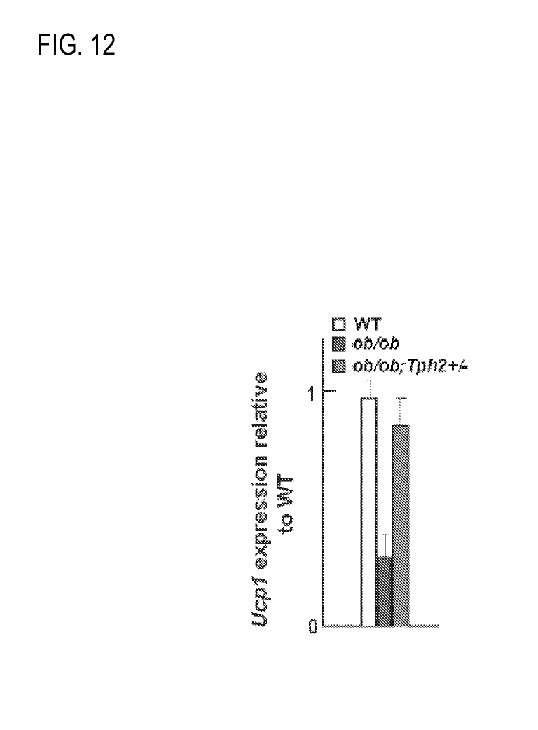

Multiple lines of evidence indicate that it is by inhibiting BDS synthesis that leptin prevents bone mass accrual. First, ObRb, the signaling form of the leptin receptor, is expressed in β-galactosidase-positive Tph2-expressing neurons (FIG. 5A). Second, Tph2 expression increased steadily over time in ob/ob mice to eventually reach a level 10 fold higher than what is seen in WT mice at 6 months of age (FIG. 5B); conversely, serotonin content is significantly higher in the brainstem of ob/ob mice (FIG. 5C). Third, leptin ICV infusion decreased Tph2 expression in a time- and dose-dependent manner in WT mice (FIG. 5D-E). Fourth, co-immunolocalization studies revealed that the phosphorylation of Stat3, a transcription factor mediating leptin signaling that was increased in β-galactosidase-positive serotonergic neurons of the brainstem following acute leptin ICV infusion in WT mice, was dramatically reduced in ObRb_{SERT}−/− mice (FIG. 5F). In support of these correlative arguments, ob/ob mice lacking one allele of Tph2 (ob/ob; Tph2+/− mice) displayed normal Tph2 expression, normal serotonin content in the brainstem, normal sympathetic tone and normal bone remodeling parameters and bone mass (FIG. 5G-I and FIG. 12). These data suggest a model whereby leptin regulates bone mass accrual through a double inhibitory loop. Leptin inhibits synthesis of BDS, which in turn reduces, by signaling in VMH neurons, the sympathetic tone; as a result leptin prevents bone mass accrual.

Certain embodiments of the invention are directed to raising bone mass accrual in a patient having lower than desired bone mass by administering a therapeutically effective amount of a leptin receptor blocker, alone or together with an Htr2c agonist.

### Therapeutic Agents

TPH2 inhibitors include p-Chlorophenylalanine Compound Action CAS number [7424-00-2] available from Tocris Bioscience, and rifampin.

Htr1a, 2b and 2c receptor antagonists further include antibodies or antibody fragments or variants thereof that bind to and reduce activity of the targeted receptor.

Agonists and Antagonists of the Htr2c Receptor

Agonists of the Htr2c receptor include m-chlorophenylpiperazine (mCPP); Kahn, R. S, and Wetzler, S., 1991. m-Chlorophenylpiperazine as a probe of serotonin function. Biol Psychiatry 30, pp. 1139-1166; Moss, H. B., Yao, J. K. and Panzak, G. L., 1990. Serotonergic responsivity and behavioral dimensions in antisocial personality disorder with substance abuse Biol Psychiatry 28, pp. 325-338; and Jaakko Lappalainena, Jeffrey C. Longa, Matti Virkkunend, Norio Ozakib, David GoldmanCorresponding Author Contact Information, b and Markku Linnoilac, Biological Psychiatry Volume 46, Issue 6, 15 Sep. 1999, Pages 821-826. Also included are (+/−)-1-(4-iodo-2,5-dimethoxy-phenyl)-2-aminopropane; 1-(3-chlorophenyl)piperazine; desyrel; nefazodone; tradozone; 1-(alpha,alpha,alpha-trifluoro-m-tolyl)-piperazine; (dl)-4-bromo-2,5-dimethoxyamphetamineHCl; (dl)-2,5-dimethoxy-4-methylamphetamine HCl; quipazine; and 6-c35. hloro-2-(1-piperazinyl)pyrazine. Among the 5-HT2 agonists, the most extensively studied is the 1-(4-iodo-2,5-dimethoxyphenyl)-2-aminopropane (3 R(−)-DOI).

Htr2c receptor antagonists include 204741 and RS 102221 (Barnes and Sharp, 1999 Neuropharmacology; McCarthy et al., 2005 Human genetics).

### CREB Antagonists

CREB antagonists include: ICER (Jaworski et al. 2003 Journal of neuroscience) and CREB-M1 (Dworkin et al., 2007 Developmental biology).

### Agonists and Antagonists of the Serotonin Htr1a Receptor

An example of an Htr1a agonist is [3H]-8-OH-DPAT (8-hydroxy-2-(di-n-propylaminotetralin)

Antagonists of the serotonin Htr1a receptor suitable for use in the methods of suppressing appetite, reducing body weight, or treating obesity disclosed herein include, but are not limited to, the following:

AP159 (N-cyc lohexyl-1,2,3,4-tetrahydrobenzo[b)thieno(2,3c)pyridine]-3-carboamide, hydrochloride), see Nagatani et al., 1991, Psychopharmacology (Berl).104(4):432-438.

Robalzotan ((R)-3-N,N-dicyclobutylamino-8-fluoro-3,4-dihydro-2H-1-benzopyran-5-carboxamide), see Muckem 2000, Curr. Opin. Investig. Drugs 1(2):236-240; Johansson et al., 1997, J. Pharmacol. Exp. Ther. 283(1):216-225. Robalzotan is particularly useful in the methods of the present invention as the hydrogen-tartrate monohydrate salt [(R)-3-N,N-dicyclobutylamino-8-fluoro-3,4-dihydro-2H-1-benzopyran-5-carboxamide hydrogen (2R,3R)-tartrate monohydrate].

WAY 100635 (N-(2-(1-(4-(2-methoxyphenyl)piperazin-yl))ethyl)-N-(2-pyridinyl)cyclohexanecarboxamide)

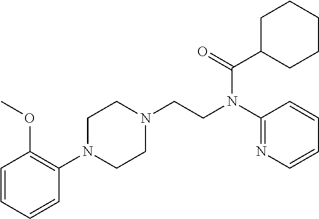

see Misane & Ogren, 2003, Neuropsychopharmacology 28:253-264; Critchley et al., 1994, Eur. J. Pharmacol. 264:95-97. WAY 100635 is particularly useful in the methods of the present invention as the trihydrochloride salt.

BMY 7378 (8-[2-[4-(2-methoxyphenyl)-1-piperazinyl]ethyl]-8-azaspiro[4,5]decane-7,9-dione dihydrochloride) (Sathi et al., 2008, Eur. J. Pharmacol. 584:222-228; Yocca et al., 1987, Eur. J. Pharmacol. 137:293-294).

Spiroxatrine (8-(2,3-dihydro-1,4-benzodioxin-2-ylmethyl)-1-phenyl-1,3,8-triazaspiro[4.5]decan-4-one)

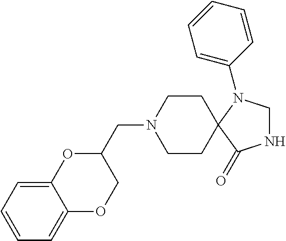

see Barrett et al., 1989, Psychopharmacology (Berl) 97:319-325; Nelson & Taylor, 1986, Eur. J. Pharmacol. 124:207-208.

Rec 15-3079 (N-[2-[4-(2-Methoxyphenyl)-1-piperazinyl]ethyl]-N-(2-nitrophenyl)cyclohexanecarboxamide), see Leonardi et al., 2001, J. Pharmacol. Exp. Ther. 299:1027-1037.

DU-125530 (2-[4-[4-(7-Chloro-2,3-dihydro-1,4-benzdioxyn-5-yl)-1-piperazinyl]butyl]-1,2-benzisothiazol-3-(2H)-one-1,1-dioxide), see Rabiner et al., 2002, J. Pharmacol. Exp. Ther. 301:1144-1150.

Lecozotan (4-cyano-N-{2R-[4-(2,3-dihydrobenzo[1,4]-dioxin-5-yl)-piperazin-1-yl]-propyl}-N-pyridin-2-yl-benzamide HCl)

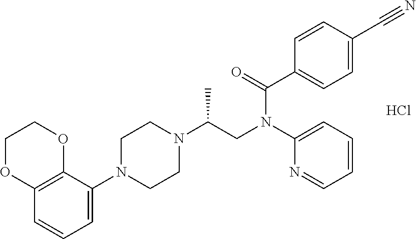

see Childers et al., 2005, J. Med. Chem. 48:3467-3470; Schechter et al., 2005, J. Pharmacol. Exp. Ther. 314:1274-1289.

Indorenate (5-methoxytryptamine beta-methylcarboxylate)

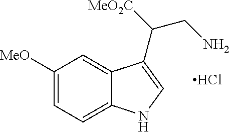

see Schoeffter & Hoyer, 1988, Br. J. Pharmacol. 95:975-985; Fernandez-Guasti & López-Rubalcava, 1990, Psychopharmacology (Berl). 101:354-358.

S-14489 [4-(benzodioxan-5-yl)1-[2-(benzocyclobutane-1-ypethyl]piperazine], see Milan et al., 1994, J. Pharmacol. Exp. Ther. 268:337-352.

S-15535 [4-(benzodioxan-5-yl)1-(indan-2-yDpiperazine)], see Millan et al., 1994, J. Pharmacol. Exp. Ther. 268:337-352.

S-15931 [4-(benzodioxan-5-yl)1-[2(indan-1-yl)ethyl]piperazine], see Millan et al., 1994, J. Pharmacol. Exp. Ther. 268:337-352.

SDZ 216-525 [methyl 4-(4-[4-(1,1,3-trioxo-2H-1,2-benzoisothiazol-2-yl)butyl]-1-piperazinyl)1H-indole-2-carboxylate] see Schoeffter et al., 1993, Eur. J. Pharmacol. 244:251-257.

Tertatolol [d,l-hydroxy-2′-t-butylamino-3′ propyloxy-8-thiochromane HCl]

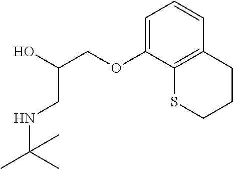

see Jolas et al., 1993, Naunyn. Schmiedebergs Arch. Pharmacol. 347:453-463.

EF-7412 [2-[4-[4-(m-(ethylsulfonamido)-phenyl)piperazin-1-yl]butyl]-1,3-dioxoperhydropyrrolo[1,2-c]imidazole], see López-Rodríguez et al., 1999, Bioorg. Med. Chem. Lett. 9:1679-1682.

Methiothepin

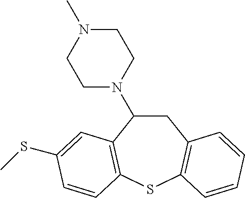

see Boddeke et al., 1992, Naunyn. Schmiedebergs Arch. Pharmacol. 345:257-263.

Pindolol

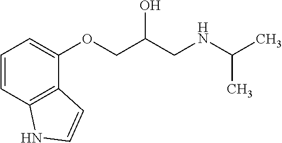

see Boddeke et al., 1992, Naunyn. Schmiedebergs Arch. Pharmacol. 345:257-263.

Compounds having the formula

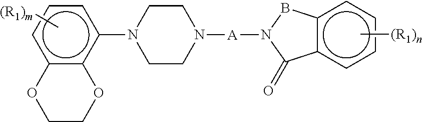

wherein R₁ is halogen, lower alkyl or alkoxy, hydroxy, trifluoromethyl or cyano,

m has the value 1 or 2 and n has the value 0 or 1,

A represents an alkylene chain containing 2-6 C-atoms which may be substituted with one more lower alkyl groups or a monocyclic (hetero)aryl group, and

B is methylene, ethylene, carbonyl, sulfinyl, sulfonyl, or sulfur.

See U.S. Pat. No. 5,462,942.

Also included is 4-amino-2-(hetero)aryl-butanamides disclosed in U.S. Pat. No. 5,610,295.

### Agonists and Antagonists of the Serotonin Htr2b Receptor

Agonists of the serotonin Htr2b receptor include BW 723C86; Papageorgiou A, Denef C., “Endocrinology. 2007 September; 148(9):4509-22. Epub 2007 Jun. 21.

Antagonists of the serotonin Htr2b receptor suitable for use in the methods of suppressing appetite, reducing body weight, or treating obesity disclosed herein include, but are not limited to, the following:

Compounds having the formula

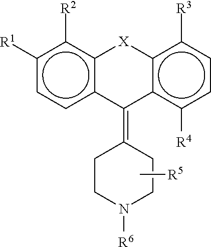

wherein R¹ is selected from the group consisting of ethyl, propyl, isopropyl, butyl, isobutyl, pentyl, hexyl, ethoxy, propoxy, isopropoxy, butoxy, isobutoxy, phenoxy, trifluoromethyl, trifluoromethoxy, amino, dimethylamino, —CON(CH₃)₂ and —CON(C₂H₅)₂;
R² is a methyl, ethyl, propyl, isopropyl, butyl, isobutyl, pentyl, hexyl, hydroxy or hydrogen, or R¹ and R² together form a five-membered heterocycle, wherein a heteroatom in said heterocycle is an oxygen atom;
R³ is selected from the group consisting of methyl, ethyl, propyl, isopropyl, butyl isobutyl, pentyl, hexyl, hydroxy and hydrogen;
R⁴ is selected from the group consisting of hydroxy, methoxy, ethoxy, propoxy, isopropoxy, butoxy, isobutoxy, trifluoromethyl, amino, dimethylamino, diethylamino, fluorine, chlorine, bromine, methyl, ethyl, propyl, isopropyl, butyl and hydrogen;
R⁵ is methyl or hydrogen;
R⁶ is methyl or ethyl; and

### X is S, N or Se;

provided that when R¹ is ethoxy and X is S, at least one of R², R³, Wand R⁵ is not hydrogen. See also U.S. Pat. No. 7,060,711.
SB 224289 (Papageorgiou and Denef, 2007 Endocrinology) is also an Htr2b receptor antagonist.

### Pharmaceutical Compositions

Therapeutic agents including the serotonin Htr1a antagonists and agonists, Htr2b antagonists and agonists and Htr2c agonists and antagonists; the leptin receptor agonists and antagonists; and the Tph2 inhibitors disclosed herein may be formulated into pharmaceutical compositions. The therapeutic agents may be present in the pharmaceutical compositions in the form of salts of pharmaceutically acceptable acids or in the form of bases. The therapeutic agents may be present in amorphous form or in crystalline forms, including hydrates and solvates. Preferably, the pharmaceutical compositions comprise a therapeutically effective amount of the Htr1a antagonists, Htr2b antagonists, and Tph2 inhibitors disclosed herein.

Pharmaceutically acceptable salts of the therapeutic agents described herein include those salts derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate salts. Other acids, such as oxalic, while not in themselves pharmaceutically acceptable, may be employed in the preparation of salts useful as intermediates in obtaining pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the therapeutic agents disclosed herein. Water or oil-soluble or dispersible products may be obtained by such quaternization.

Pharmaceutically acceptable derivatives of any of the Htr1a antagonists, Htr2b antagonists, and Tph2 inhibitors disclosed herein come within the scope of the invention. A “pharmaceutically acceptable derivative” of a Htr1a antagonist, Htr2b antagonist, or Tph2 inhibitor means any non-toxic derivative of the Htr1a antagonist, Htr2b antagonist, or Tph2 inhibitor that, upon administration to a patient, exhibits that same or similar biological activity with respect to decreasing weight or suppressing appetite as the Htr1a antagonist, Htr2b antagonist, or Tph2 inhibitor.

The therapeutic agents of the present invention are also meant to include all stereochemical forms of the therapeutic agents (i.e., the R and S configurations for each asymmetric center). Therefore, single enantiomers, racemic mixtures, and diastereomers of the therapeutic agents are within the scope of the invention. Also within the scope of the invention are steric isomers and positional isomers of the therapeutic agents. The therapeutic agents of the present invention are also meant to include compounds which differ only in the presence of one or more isotopically enriched atoms. For example, therapeutic agents in which one or more hydrogens are replaced by deuterium or tritium, or the replacement of one or more carbons by ¹³C- or ¹⁴C-enriched carbon are within the scope of this invention.

In a preferred embodiment, the therapeutic agents of the present invention are administered in a pharmaceutical composition that includes a pharmaceutically acceptable carrier, adjuvant, or vehicle. The term “pharmaceutically acceptable carrier, adjuvant, or vehicle” refers to a non-toxic carrier, adjuvant, or vehicle that does not destroy or significantly diminish the pharmacological activity of the therapeutic agent with which it is formulated. Pharmaceutically acceptable carriers, adjuvants or vehicles that may be used in the compositions of this invention encompass any of the standard pharmaceutically accepted liquid carriers, such as a phosphate-buffered saline solution, water, as well as emulsions such as an oil/water emulsion or a triglyceride emulsion. Solid carriers may include excipients such as starch, milk, sugar, certain types of clay, stearic acid, talc, gums, glycols, or other known excipients. Carriers may also include flavor and color additives or other ingredients.

The pharmaceutical compositions of the present invention are preferably administered orally, preferably as solid compositions. However, the pharmaceutical compositions may be administered parenterally, by inhalation spray, topically, rectally, nasally, buccally, vaginally or via an implanted reservoir. Sterile injectable forms of the pharmaceutical compositions may be aqueous or oleaginous suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The pharmaceutical compositions employed in the present invention may be orally administered in any orally acceptable dosage form, including, but not limited to, solid forms such as capsules and tablets. In the case of tablets for oral use, carriers commonly used include microcrystalline cellulose, lactose and corn starch. Lubricating agents, such as magnesium stearate, are also typically added. When aqueous suspensions are required for oral use, the active ingredient may be combined with emulsifying and suspending agents. If desired, certain sweetening, flavoring or coloring agents may also be added.

The pharmaceutical compositions employed in the present invention may also be administered by nasal aerosol or inhalation. Such pharmaceutical compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Should topical administration be desired, it can be accomplished using any method commonly known to those skilled in the art and includes but is not limited to incorporation of the pharmaceutical composition into creams, ointments, or transdermal patches.

The pharmaceutical compositions employed in the present invention can be formulated to increase delivery of the Htr1a antagonists, Htr2b antagonists, or Tph2 inhibitors to the central nervous system. If an Htr1a antagonist, Htr2b antagonist, or Tph2 inhibitor having therapeutic utility does not easily cross the blood brain barrier, various methods known in the art can be employed to improve permeability through the blood brain barrier.

The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier. The passage of the agent can also be facilitated by its covalent coupling to a peptide vector capable of transporting the agent through the blood-brain barrier. Peptide transport vectors known as blood-brain barrier permeabilizer compounds are disclosed in U.S. Pat. No. 5,268,164. Site specific macromolecules with lipophilic characteristics useful for delivery to the brain are disclosed in U.S. Pat. No. 6,005,004.

Additional therapeutic agents, which are normally administered to control weight or appetite may also be present in the pharmaceutical compositions employed in the present invention. Examples of appropriate agents include catecholamines, lipase inhibitors, sibutramine, orlistat, and rimonabant. Those additional agents may be administered separately from the Htr1a antagonists, Htr2b antagonists, and Tph2 inhibitors disclosed herein, as part of a multiple dosage regimen. Alternatively, those agents may be part of a single dosage form, mixed together with the Htr1a antagonists, Htr2b antagonists, and Tph2 inhibitors disclosed herein in a single pharmaceutical composition. If administered as part of a multiple dosage regimen, the two active agents may be administered simultaneously, sequentially or within a pre-selected period of time from one another. The amount of both the Htr1a antagonists, Htr2b antagonists, and Tph2 inhibitors disclosed herein and the additional therapeutic agent that may be combined with the carrier materials to produce a single dosage form will vary depending upon the host treated and the particular mode of administration as well as on the nature of the Htr1a antagonists, Htr2b antagonists, and Tph2 inhibitors disclosed herein and the additional therapeutic agent.

In certain embodiments, the present invention provides methods where a patient is administered either an antagonist of the serotonin Htr1a receptor, an antagonist of the serotonin Htr2b receptor, or a Tph2 inhibitor and no other active pharmaceutical ingredient. In some embodiments, the patient is administered no other substance known to be effective for the treatment of eating disorders other than the antagonist of the serotonin Htr1a receptor, the antagonist of the serotonin Htr2b receptor, or the Tph2 inhibitor.

### Dosages

The amount of the Htr1a antagonist or agonist, Htr2b antagonist or agonist, Htr2c agonist or antagonist or Tph2 inhibitor that may be combined with carrier materials to produce a pharmaceutical composition in a single dosage form will vary depending upon the patient treated and the particular mode of administration. It should be understood that a specific dosage and treatment regimen for any particular patient will depend upon a variety of factors, including the activity of the specific Htr1a antagonist, Htr2b antagonist, or Tph2 inhibitor employed, the age, body weight, general health, sex, diet, time of administration, rate of excretion, drug combination, and the judgment of the treating physician as well as the severity of the particular condition being treated. Despite their variety, accounting for these factors in order to select an appropriate dosage or treatment regimen would require no more than routine experimentation.

The amount of Htr1a antagonist/agonist, Htr2b antagonist/agonist, Htr2c agonist/antagonist, or Tph2 inhibitor to be administered in the present invention depends on many factors, as discussed above. However, in humans, for example, the amount ranges from about 1 mg/day to about 2 g/day; preferably from about 15 mg/day to about 500 mg/day; or from about 20 mg/day to about 250 mg/day; or from about 40 mg/day to about 100 mg/day. Other preferred dosages include about 2 mg/day, about 5 mg/day, about 10 mg/day, about 15 mg/day, about 20 mg/day, about 25 mg/day, about 30 mg/day, about 40 mg/day, about 50 mg/day, about 60 mg/day, about 70 mg/day, about 80 mg/day, about 90 mg/day, about 100 mg/day, about 125 mg/day, about 150 mg/day, about 175 mg/day, about 200 mg/day, about 250 mg/day, about 300 mg/day, about 350 mg/day, about 400 mg/day, about 500 mg/day, about 600 mg/day, about 700 mg/day, about 800 mg/day, and about 900 mg/day. Routine experimentation will determine the appropriate value for each patient by monitoring the effect of the therapeutic agent(s) on patient weight or appetite, which can be frequently and easily monitored. The Htr1a antagonist, Htr2b antagonist, or Tph2 inhibitor can be administered once or multiple times per day. The frequency of administration may vary from a single dose per day to multiple doses (1, 2, 3, 4, or more) per day. The daily dosage regimen will preferably be from 0.01 to 200 mg/kg, 0.05 to 175 mg/kg, 0.1 to 150 mg/kg, 0.5 to 100 mg/kg, pr 1 to 75 mg/kg, of total body weight.

In certain embodiments, the Htr1a antagonist/agonist, Htr2b antagonist/agonist, Htr2c antagonist/agonist, Tph2 inhibitor or combinations thereof are repeatedly administered to the patient and the patient's appetite and/or weight is measured until it is reduced to a desired level. For example, in certain embodiments, the patient's weight is reduced by at least about 3%, 5%, 10%, 15%, or 20% compared to the patient's weight prior to the first administration of the Htr1a antagonist, Htr2b antagonist, or Tph2 inhibitor.

### Assays for Identifying Htr1a Antagonists, Htr2b Antagonists, and Tph2 Inhibitors

In addition to the specific Htr1a antagonists, Htr2b antagonists, and Tph2 inhibitors disclosed herein, the methods of the present invention may be practiced using additional Htr1a antagonists, Htr2b antagonists, and Tph2 inhibitors that may be identified by methods known in the art or by the methods disclosed herein.

In certain embodiments, the Htr1a antagonist, Htr2b antagonist, or Tph2 inhibitor that is identified may be a small organic molecule, an antibody, an antibody fragment, a protein, or a polypeptide. Preferably, the Htr1a antagonist, Htr2b antagonist, or Tph2 inhibitor is a small organic molecule. By “small organic molecule” is meant an organic compound of molecular weight of more than 100 and less than about 2,500 daltons, and preferably less than 500 daltons.

Antagonists of the serotonin Htr1a, Htr2b or Htr2c receptor may be identified by a method comprising:

(a) providing a cell expressing the desired target serotonin receptor (Htr1a, Htr2b or Htr2c;
(b) exposing the cell of step (a) to serotonin or an agonist of the desired serotonin receptor in the absence of a candidate compound;
(c) measuring the activation of the desired target serotonin receptor in the cell of step (b) in the absence of the candidate compound;
(d) exposing the cell expressing the desired target serotonin receptor to serotonin or an agonist of the desired target serotonin receptor in the presence of the candidate compound;
(e) measuring the activation of the desired target serotonin receptor in the cell of step (d) in the presence of the candidate compound; and
(f) if the amount of activation of the desired target serotonin receptor in the measured in step (e) is less than the amount of activation of the desired target serotonin receptor measured in step (c) in the absence of the candidate compound, then determining that the candidate compound is an antagonist of the desired target serotonin receptor.

In certain embodiments, where the desired target serotonin receptor is Htr1a or Htr2b, the method described above includes the further step of administering the respective serotonin receptor antagonist identified in step (f) to a patient in need of therapy for an eating disorder, e.g., obesity. In certain embodiments, a decrease in appetite or body weight of the patient is observed after administration of the serotonin receptor antagonist identified in step (f) to the patient.

Candidate compounds may be screened directly from a collection of candidate compounds by the above method or candidate compounds may be first tested for the ability to displace the binding of a known ligand of the desired targeted serotonin receptor (Htr1a, Htr2b or Htr1c) by a method comprising:

(a) providing a cell expressing the serotonin desired target receptor;
(b) exposing the cell expressing the desired target serotonin receptor to serotonin or an agonist of the desired target serotonin receptor in the absence of a candidate compound;
(c) measuring the binding of serotonin or the serotonin receptor agonist to the desired target serotonin receptor in the cell of step (b) the absence of the candidate compound;
(d) exposing the cell of step (b) to serotonin or an agonist of the desired target serotonin receptor in the presence of a candidate compound;
(e) measuring the binding of the serotonin or the serotonin receptor agonist to the desired target serotonin receptor in the cell of step (d) in the presence of the candidate compound;
(f) where, if the binding of the serotonin or the serotonin receptor agonist to the desired target serotonin receptor in the cell of step (d) in the presence of the candidate compound is less than the binding of the serotonin or the desired target serotonin receptor agonist to the desired target serotonin receptor in the cell of step (b) in the absence of the candidate compound, the candidate compound is able to displace the binding of a known ligand of the desired target serotonin receptor.

In certain embodiments, where the desired target serotonin receptor is a Htr1a or 2b receptor, the method described above includes the further step of administering the candidate compound identified in step (f) that is able to displace the binding of a known ligand of the desired target serotonin receptor to a patient in need of therapy for an eating disorder, e.g., obesity. In certain embodiments, a decrease in appetite or body weight of the patient is observed after administration of the candidate compound.

To facilitate measuring the binding of the serotonin or the agonist of the serotonin receptor to the cells in steps (b) and (d) above, either the serotonin or the agonist may be suitably labeled.

Assays for discovering Htr1a antagonists may be based on the ability to competitively displace the binding of the labeled serotonin Htr1a receptor agonist [³H]-8-OH-DPAT (8-hydroxy-2-(di-n-propylaminotetralin) at serotonin Htr1a receptors (Millan et al., 1994, J. Pharmacol. Exp. Ther. 268:337-352). In certain embodiments, the new antagonists will be selected from those compounds that exhibit binding affinities to the serotonin Htr1a receptor (pKᵢs) of 10 μM or less.

Assays for discovering Htr1a, Htr2b or Htr1c antagonists may be carried out in HeLa cell lines that have been transfected with and express the respective desired target human receptor. Binding of candidate antagonists to the respective human receptor may be determined by displacement of a radiolabeled ligand of the desired target receptor. For Htr1a, this ligand may be [³H]8-OH-DPAT. The functional activity of a candidate Htr1a antagonists may be assayed by effects on the calcium response (measured using Fura-2) (Boddeke et al., 1992, Naunyn. Schmiedebergs Arch. Pharmacol. 345:257-263). The partial wild type sequence of the human Htr1a receptor has been disclosed in Parks & Shenk, 1996, J. Biol. Chem. 271:4417-4430.

Assays for discovering Htr1a antagonists may be carried out by testing candidate compounds for the ability to displace [³H]8-OH-DPAT from specific binding sites in rat frontal cortex homogenates (Gozlan et al., 1983, Nature 305:140-142). Candidate compounds may also be tested for Htr1a receptor binding activity in rat hippocampal membrane homogenates (Alexander & Wood, 1988, J. Pharm. Pharmacol. 40:888-891). Candidate compounds may also be tested for Htr1a receptor antagonist activity in a test involving the antagonism of 5-carboxamidotryptamine in the guinea-pig ileum in vitro (Fozard et al., 1985, Br. J. Pharmacol. 86:601 P).

Compounds identified as Htr1a antagonists by in vitro assays such as those described above may be further tested for their in vivo Htr1a antagonist activity, e.g., by determining whether such compounds can antagonize 8-OH-DPAT-induced effects in rats, e.g., antagonism of hypothermia or lower lip retraction (Broekkamp et al., 1989, Pharmacol. Biochem. Behav. 33:821-827).

Inhibitors of Tph2 may be identified by any methods known in the art. In particular, inhibitors of Tph2 may be identified by a method comprising:

(a) providing a source of Tph2;
(b) exposing the source of Tph2 to L-tryptophan in the absence of a candidate compound;
(c) measuring the amount of 5-hydroxytryptophan produced by the source of Tph2 in the absence of the candidate compound;
(d) exposing the source of Tph2 to L-tryptophan in the presence of the candidate compound;
(e) measuring the amount of 5-hydroxytryptophan produced by the source of Tph2 in the presence of the candidate compound;
(f) where, if the amount of 5-hydroxytryptophan produced by the source of Tph2 in the presence of the candidate compound is less than the amount of 5-hydroxytryptophan produced by the source of Tph2 in the absence of the candidate compound, the candidate compound is identified as a Tph2 inhibitor.

In certain embodiments, the method described above includes the further step of administering the Tph2 inhibitor identified in step (f) to a patient in need of therapy for an eating disorder, e.g., obesity. In certain embodiments, a decrease in appetite or body weight of the patient is observed after administration of the Tph2 inhibitor identified in step (f) to the patient.

“Less than” for the purpose of the herein-described methods of identifying therapeutic agents from a collection of candidate compounds refers to an amount that would not be attributed by those of skill in the art to normal variation seen in the method. Preferably, “less than” is at least about 10%, at least about 20%, at least about 50%, at least about 75%, or at least about 95% less than the amount observed in the absence of the candidate compound.

In certain embodiments, the source of Tph2 is an isolated Tph2 enzyme, preferably human. Isolated Tph2 can be produced by in vitro expression of Tph1, e.g., in a coupled in vitro transcription/translation system. Alternatively, the source of Tph2 may be partially or highly purified preparations from cells expressing Tph2. In other embodiments, the source of Tph2 is a whole cell expressing Tph2, preferably human. In some embodiments, the whole cell has been transfected with a expression vector comprising Tph2 so that the cell expresses recombinant Tph2, preferably human.

The mRNA and amino acid sequence of human Tph2 can be found in GenBank, at accession no. AY098914. The genomic sequence can be found at AC090109. These nucleotide sequences can be used in methods well-known in the art to construct suitable expression vectors for expressing Tph2 recombinantly in cells, or in vitro.

In certain embodiments, the present invention provides a method of treating eating disorders, suppressing appetite, reducing body weight, and treating obesity by the administration of an antagonist of the serotonin Htr1a receptor, an antagonist of the serotonin Htr2b receptor, or a Tph2 inhibitor, or combinations thereof to a patient known to be in need of treatment for the eating disorder, suppression of appetite, reduction of body weight, or treatment for obesity comprising:

(a) providing a plurality of candidate compounds;
(b) determining that one of the plurality of candidate compounds is an antagonist of the serotonin Htr1a receptor, an antagonist of the serotonin Htr2b receptor, or a Tph2 inhibitor;
(c) administering to the patient known to be in need of treatment for the eating disorder, suppression of appetite, reduction of body weight, or treatment for obesity a therapeutically effective amount of the candidate compound determined to be an antagonist of the serotonin Htr1a receptor, an antagonist of the serotonin Htr2b receptor, or a Tph2 inhibitor in step (b).

Preferably, the antagonists of the serotonin Htr1a receptor, antagonists of the serotonin Htr2b receptor, and the Tph2 inhibitors identified by the methods described herein should be capable of crossing the blood-brain barrier. Alternatively, methods known in the art for delivery substances across the blood-brain barrier may be employed to deliver those antagonists of the serotonin Htr1a receptor, antagonists of the serotonin Htr2b receptor, and the Tph2 inhibitors identified by the methods described herein that are not capable of crossing the blood-brain barrier.

### Derivatives and Prodrugs of Htr1a Antagonists, Htr2b Antagonists, and Tph2 Inhibitors

The Htr1a antagonists, Htr2b antagonists, and Tph2 inhibitors used in the present invention include derivatives and/or prodrugs. Accordingly, the present invention also encompasses the use of certain derivatives of the Htr1a antagonists, Htr2b antagonists, and Tph2 inhibitors disclosed herein. For example, prodrugs of the Htr1a antagonists, Htr2b antagonists, and Tph2 inhibitors could be produced by esterifying the carboxylic acid functions of the Htr1a antagonists, Htr2b antagonists, and Tph2 inhibitors with a lower alcohol, e.g., methanol, ethanol, propanol, isopropanol, butanol, etc. The use of prodrugs of the Htr1a antagonists, Htr2b antagonists, and Tph2 inhibitors that are not esters is also contemplated. For example, pharmaceutically acceptable carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters of the Htr1a antagonists, Htr2b antagonists, and Tph2 inhibitors are also contemplated. In some embodiments, the prodrugs will contain a biohydrolyzable moiety (e.g., a biohydrolyzable amide, biohydrolyzable carbamate, biohydrolyzable carbonate, biohydrolyzable ester, biohydrolyzable phosphate, or biohydrolyzable ureide analog). Guidance for the preparation of prodrugs of the Htr1a antagonists, Htr2b antagonists, and Tph2 inhibitors disclosed herein can be found in publications such as Design of Prodrugs, Bundgaard, A. Ed., Elsevier, 1985; Design and Application of Prodrugs, A Textbook of Drug Design and Development, Krosgaard-Larsen and H. Bundgaard, Ed., 1991, Chapter 5, pages 113-191; and Bundgaard, H., Advanced Drug Delivery Review, 1992, 8, pages 1-38.

In certain embodiments of the invention, a therapeutically effective amount of one or more of the Htr1a antagonists, Htr2b antagonists, or Tph2 inhibitors is administered in combination with another weight-loss drug or appetite suppresant. Two classes of such drugs are the intestinal lipase inhibitor class, which reduce fat digestion and absorption, and the centrally acting mixed norepinephrine/serotonin reuptake blockers, e.g., sibutramine. Suitable examples of agents for treating obesity include appetite suppressants such as benzphetamine, diethylpropion, Mazindol, phendimetrazine and phentermine.

Optionally, a therapeutically effective amount of one or more of the Htr1a antagonists, Htr2b antagonists, or Tph2 inhibitors is administered in combination with additional agents that include but are not limited to compounds which are known to treat obesity related disorders such as diabetes. Examples of agents for treating diabetes include insulin for insulin-dependent diabetes (IDDM) and sulfonylurea compounds for non-insulin dependent diabetes (NIDDM). Examples of sulfonylureas include tolbutamide, chlorpropamide, tolazamide, acetohexamide, glycburide, glipizide and gliclazide.

The present invention encompasses the use of an Htr1a antagonist, an Htr2b antagonist, or a Tph2 inhibitor, or combinations thereof, for the manufacture of a medicament for treating eating disorders, suppressing appetite, reducing body weight, or treating obesity. The present invention encompasses the use of an Htr1a antagonist, an Htr2b antagonist, or a Tph2 inhibitor for treating eating disorders, suppressing appetite, reducing body weight, or treating obesity.

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference as if set forth herein in their entirety, except where terminology is not consistent with the definitions herein. Although specific terms are employed, they are used as in the art unless otherwise indicated.

### EXAMPLES

### Example 1

### A. Mice Generation

Tph2-LacZ mice were generated by embryonic stem cell manipulations following standard protocols to obtain Tph2+/− mice. Tph2+/− mice were intercrossed to obtain the WT, Tph2+/− and Tph2−/− mice for analysis. Generation of Tph1−/−, Htr2c−/−, loxTB Htr2c, Htr1α−/−, ObR^{fl/fl}, Htr2b^{fl/fl}, Sf1-Cre and Sert-Cre mice was previously reported (Balthasar et al., 2004, Neuron 42:983-991; Dhillon et al., 2006, Neuron 49:191-203; Klemenhagen et al., 2006, Neuropsychopharmacology 31:101-111; Tecott et al., 1995, Nature 374:542-546; van de Wall et al., 2008, Endocrinology 149:1773-1785; Xu et al., 2008, Neuron 60:582-589; Yadav et al., 2008, Cell 135:825-837; Zhuang et al., 2005, J. Neurosci. Methods. 143:27-32). WT, Pomc1-Cre and ob/ob mice were obtained from The Jackson Laboratory.

To generate mice lacking Htr1a, Htr2b, Creb in Pomc-expressing neurons flox/+ mice were crossed with Pomc-Cre mice (obtained from Jackson laboratories), and their progeny was intercrossed to obtain Htr1a_{Pomc}−/−, Htr2b_{Pomc}−/−, Htr1a; 2b_{Pomc}−/− and Creb_{Pomc}−/− mice. Generation of Htr1d^{fl/fl}, Htr2b^{fl/fl} and Creb^{fl/fl} was previously reported (Yadav et al, Cell 2008; Heath and Hen, 1995; Weisstaub et al., 2006). Wild-type C57 B16/J, ob/ob mice were obtained from the Jackson laboratories. All experiments were conducted following Columbia University Guidelines for the Animal Use and Care of laboratory mice.

### B. Experimental Regimen for Food Intake Measurement

### 1. WT and Mutant Animals

Animals were housed under 12 h light/12 h dark conditions with ad libitum access to food and water, and were used after a minimum of 4 days of acclimatization to the housing conditions. Control, Htr1a_{Pomc}−/−, Htr2b_{Pomc}−/−, Htr1a; 2 b_{Pomc}−/−, Creb_{Pomc}−/− mice were separated into individual cages one day prior to the experiment. Food intake and energy expenditure was measured every 12 hours for 36 hours essentially as described previously (6, 17).

2. WT and ob/ob Mice Treated with Vehicle or Htr1a Antagonist LY426965

One or 3-month old C57B1/6J inbred female mice were used in these experiments. Two different experimental regimens were utilized to assess the effect of LY426965 on appetite in WT and ob/ob mice.

### Example 2

### Histological Procedures, Immunohistochemistry, In Situ Hybridization, Axonal Tracing and Microcomputed Tomography (μCT) Analysis

Sections containing dorsal raphe were from bregma −4.04 to −5.40; median raphe from −4.04 to −4.48; caudal raphe from −4.84 to −7.48; arcuate from −1.22 to −2.80; VMH from −1.06 to −2.06 and PVN from −0.58 to −1.22 according to Franklin and Paxinos mouse brain atlas. Immunohistochemistry was performed on paraffin-embedded specimens sectioned at 6 μm according to standard protocols. LacZ staining was performed on whole brain and coronal sections obtained from the Tph2+/− mice following standard procedures. In situ hybridization on brain sections was performed as described (Oury et al., 2006, Science 313:1408-1413). Ex vivo axonal tracing was performed using Rhodamine-conjugated dextrans (Molecular Probes, Eugene, Oregonaxonal; See supplemental methods for details). Bone histomorphometric analyses were performed on undecalcified sections using the Osteomeasure analysis system (Osteometrics, Atlanta). Trabecular bone architecture of proximal tibia was assessed using a μCT system (VivaCT 40, SCANCO Medical AG, Switzerland) as described (Shi et al., 2008, Proc. Natl. Acad. Sci. USA 105:20529-20533). Six to 12 animals were analyzed for each group.

### Example 3

### Bioassays

Serotonin levels in the brain and serum were quantified as described (Yadav et al., 2008, Cell 135:825-837). Serum level of total deoxypyridinoline (DPD) cross-links was measured using the Metra tDPD kit (Quidel Corp. San Diego, Calif.). Urinary elimination of catecholamines was measured in acidified spot urine samples by EIA (Bi-CAT, Alpco Diagnostics, Salem, N.H.) and creatinine (Metra creatinine kit, Quidel Corp. San Diego, Calif.) was used to standardize between urine samples.

### Example 4

### Molecular Studies

RNA isolation, cDNA preparation and qPCR analysis was carried out following standard protocols. Genotypes of all the mice were determined by PCR. All primer sequences for genotyping and DNA probes for southern hybridization are available upon request.

### Example 5

### Electrophysiology

Brain slice preparation and electrophysiological recordings were performed as reported previously (Rao et al., 2007, J. Clin. Invest. 117:4022-4033; Rao et al., 2008, J. Neurosci. 28:9101-9110). Briefly, WT and ObRbSERT−/− mice were anesthetized with ether and then decapitated. The brains were rapidly removed and immersed in an oxygenated bath solution at 4° C. containing (in mM): sucrose 220, KCl 2.5, CaCl₂ 1, MgCl₂ 6, NaH₂PO₂ 1.25, NaHCO₃ 26, and glucose 10, pH 7.3 with NaOH. Coronal slices (350 μm thick) containing dorsal raphe (DR) were cut on a vibratome and maintained in a holding chamber with artificial cerebrospinal fluid (ACSF) (bubbled with 5% CO₂ and 95% O₂) containing (in mM): NaCl 124, KCl 3, CaCl₂ 2, MgCl₂ 2, NaH₂PO₄ 1.23, NaHCO₃ 26, glucose 10, pH 7.4 with NaOH, and were transferred to a recording chamber constantly perfused with bath solution (33° C.) at 2 ml/min after at least a 1 hr recovery.

Whole-cell current clamp was performed to observe action potentials in DR seritonergic (5-HT) neurons with a Multiclamp 700A amplifier (Axon instrument, CA). The patch pipettes with a tip resistance of 4-6 MS were made of borosilicate glass (World Precision Instruments) with a Sutter pipette puller (P-97) and filled with a pipette solution containing (in mM): K-gluconate (or Cs-gluconate) 135, MgCl₂ 2, HEPES 10, EGTA 1.1, Mg-ATP 2, Na₂-phosphocreatine 10, and Na₂-GTP 0.3, pH 7.3 with KOH. After a giga-Ohm (GΩ) seal and whole-cell access were achieved, the series resistance (between 20 and 40 MΩ) was partially compensated by the amplifier. 5-HT neurons were identified according to their unique properties (long duration action potential, activation by norepinephrine and inhibition by serotonin itself) reported previously (Liu et al., 2002, J. Neurosci. 22:9453-9464). Under current clamp, 5-HT neurons were usually quiescent in slices because of the loss of noradrenergic inputs. The application of α1-adrenergic agonist phenylephrine (PE, 3 μM) elicited action potentials and the application of serotonin creatinine sulfate complex (3 μM) inhibited action potentials in these neurons. The effect of leptin on 5-HT neurons was examined in DR neurons responding to both PE and serotonin. Before the application of leptin, action potentials in 5-HT neurons were restored by application of PE in the bath (Liu et al., 2002, J. Neurosci. 22:9453-9464). All data were sampled at 3-10 kHz and filtered at 1-3 kHz with an Apple Macintosh computer using Axograph 4.9 (Axon Instruments). Electrophysiological data were analyzed with Axograph 4.9 and plotted with Igor Pro software (WaveMetrics, Lake Oswego, Oreg.).

### Example 6

### Statistical Analyses

Statistical significance was assessed by Student's t test or a one way ANOVA followed by Newman-Keuls test for comparison between more than 2 groups. P<0.05 was considered significant. Different letters indicate significant differences among groups.

### Statistical Analysis

Results are given as means±standard deviations. Statistical Analysis was performed by Student's t test. All panels in FIGS. 1-4 *p<0.05 versus WT or control. MANUSCRIPT

### Example 7

### Western Blot Analysis

Frozen hypothalamus samples were homogenized in the in 200-500 μl of RIPA buffer (10 mM NaPO₄, pH 7.0, 150 mM NaCl, 2 mM EDTA, 1% sodium deoxycholate, 1% NP-40, 0.1% SDS, 50 mM NaF, 200 mM Na₃VO₄, 0.1% β-mercaptoethanol, 1 mM PMSF, 4 μg/ml aprotinin, and 2 μg/ml leupeptin), and incubated on ice for 10 min with intermittent mixing before centrifugation at 15,000×g for 10 min at 4° C. The clarified lysate was recovered, aliquoted, and stored at −80° C. For western blot analysis, different amounts of proteins were resolved by 10% SDS-PAGE and electroblotted onto nitrocellulose/PVDF membrane using a wet transfer unit (Bio-Rad Laboratories, Richmond, Calif.). Nonspecific sites on the membrane were blocked using 10% BSA in TBST (20 mM Tris-HCl, pH 7.6, 150 mM NaCl, 0.1% Tween-20) by incubating overnight at 4° C. The membrane was then washed extensively in 1×TBST (three times for 5 min each at room temperature) and incubated at room temperature with primary antibodies (Santa Cruz biotechnology Inc.) specific for different proteins [1:200 for Htr2c (sc-17797), 1:100 for Htr2b (sc-15080) and 1:100 for Htr1a (sc-10801) in TBST containing 5% BSA] for 3 h at room temperature. Secondary antibodies (horseradish peroxidase labeled anti-rabbit/anti-goat/anti-mouse IgG) were used at 1:2500 dilution in TBST containing 5% BSA. The bands were then visualized using an ECL kit (NEN Life Sciences).

### Example 8

### Double Immunofluorescence Analysis on Brain Slices with pSTAT3:βGal; Tph2:ObRb and Tph2:βGal

Animals were anaesthetized and placed on a stereotaxic instrument (Stoelting) and the depth of anesthesia was determined by the animal's respiratory pattern and by pinching the animal's foot for reflex response. The dorsal part of the animal's head was shaved and prepped with betadine scrub, and 70% alcohol. The skin covering the head was then cut (approximately a 1 cm incision) and the calvaria exposed. A hole was drilled upon bregma using a 28-gauge needle. A 28 gauze needle canula (Brain infusion kit II, Alzet) was then implanted into the hole reaching the third cerebral ventricle according to the following coordinates: midline, −0.3 AP. 3 mm ventral (0 point Bregma). Using a Hamilton syringe, PBS or leptin (2 μg) was injected into the 3rd cerebral ventricle. The dorsal edges of the incision were coated with Bupivicaine 0.25% (<2 mg/kg), joined and closed with 2 sterile clips. One hour later mice were anesthetized and perfused transcardially with ice-cold saline followed by 10% neutral buffered formalin. Brains were removed and postfixed for 4 hr and then cryoprotected by overnight immersion in a 20% sucrose solution. Frozen brains were sliced in 25 μm coronal sections using a cryotome and sections were stored at −80° C. till utilized. For pSTAT3:βGal double immunofluorescence analysis, sections were dried at room temperature for 20 minutes, pretreated with 1% NaOH, 1% H₂O₂ (20 min), 0.3% glycine (10 min), 0.03% SDS (10 min), blocked in donkey serum, and then incubated in rabbit pSTAT3 (tyr705) antibody (1:100, Cell Signal Technology) and chicken βGal antibody (1:500, abcam) for 24 hr at 4° C. Sections were rinsed and incubated with a donkey anti-rabbit antibody (1:1000; Vector Laboratories) and donkey anti-chicken antibody (Cy3; Jackson immunoresearch).

For Tph2:βGal, 25 μm coronal sections were dried at room temperature, washed with PBS, blocked in donkey serum for 1 h and incubated with chicken βGal antibody (1:500 dilution, abcm) or rabbit Tph2 antibody (1:2, 500 dilution) or goat ObRb antibody (1:50 dilution, Santa Cruz biotechnology). Sections were rinsed and incubated with donkey anti-chicken antibody (Cy3, Jackson immunoresearch) or donkey anti-goat antibody (Cy2, Jackson immunoresearch) or donkey anti-rabbit antibody (Cy2, Jackson immunoresearch).

Following staining procedures, sections were mounted and coverslipped with aqueous anti-fade mounting medium for fluorescence. Staining was visualized and captured using a Zeiss fluorescent microscope.

### Example 9

### Melanocortin Sensitivity Analysis

To analyze changes in melanocortin sensitivity in Tph2−/− mice, MTII (2 μg) or saline was administered (ICV) into WT and Tph2−/− mice. 3 hours later mice were transcardially perfused with 4% PFA, brains were dissected and postfixed in 4% PFA overnight at 4° C. Following cryoprotection in 20% sucrose, brains were coronally sectioned at 30 μm thickness. For colorimetric cFos immunohistochemistry, sections were incubated for 16 hrs at 4° C. in rabbit anti-cFos antiserum (Ab-5; 1:3000 dilution; Calbiochem), incubated with biotinylated goat anti-rabbit IgG secondary antiserum (1:600; Vector laboratories) for 2 h at room temperature, and then incubated in avidin biotin complex (Vector Labs). Color was developed using Vector ABC kit.

### Example 10

### Double Fluorescent In Situ Hybridization

Cryosections were incubated with DIG-labeled 5-HT2c receptor (5-HT2cR) cRNA probe and FITC labeled Sf1-specific or FITC-labeled 5-HT2c receptor (5-HT2cR) cRNA probe and DIG-labeled Pomc specific cRNA. After stringent wash, sections were incubated with horseradish peroxidase (HRP)-conjugated anti-DIG antibody (1:1000) and labeled with Cy3 by using tyramide signal amplification (TSA) system (NEL744, PerkinElmer, USA). This was followed by quenching with 1% H₂O₂ and sections were incubated with HRP-conjugated anti-FITC antibody (1:1500) and labeled with FITC by TSA system (NEL741). Sections following staining were mounted in antifade mounting medium and visualized using a Leica fluorescent microscope.

### Example 11

### Metabolic Tests

Metabolic tests were performed at 2 months of age in WT, Tph2−/− and ObRbSERT−/− mice following previously published procedures (Lee et al., 2007, Cell 130:456-469). Briefly, glucose tolerance tests (GTT) were performed after 6 hours fasting. 1 g/kg of glucose was administrated in mice through an i.p. injection, and blood glucose was measured at 0, 15, 30, 60 and 120 minutes using an Accu-check glucometer (Roche). Insulin tolerance tests (ITT) were performed after 6 hours of fasting. Insulin (Sigma; 0.5 units/kg) was injected i.p. and blood glucose was measured at 0, 15, 30, 60, 90 and 120 minutes. Feeding glucose levels were measured in the morning on the mice with ad libitum access to food and water.

### Example 12

### Rhodamine-Conjugated Dextrans Labeling

Rhodamine-conjugated dextrans (Molecular Probes, Eugene, Oregonaxonal) were used as axonal tracers in an ex vivo preparation. These substances are efficiently taken up by injured axons and transported rapidly along the axonal structure anterogradely to the axonal terminals and retrogradely to the cell bodies. Anterograde and retrograde labeling were employed, respectively, for the staining of axonal projections of the Median Raphe nuclei (MR) and axonal projections reaching the VMH and Arcuate nuclei as previously described (Oury et al., 2006, Science 313:1408-1413) with the following modifications. Axonal projection of the MR nuclei were labeled by applying dextran crystals in a surgically created pouch in Tph2LacZ/+ mice (P0-P4). The surgical application of dextran was confirmed by comparison on section after β-galactosidase staining visualizing the serotonergic neurons. Axonal tracing of the projections reaching the VMH and Arcuate nuclei were performed in Sf1-Cre/Rosa26R and Pomc-Cre/Rosa26R mice respectively and realized in Tph2LacZ/+ mice by applying dextran crystals in the hypothalamus between the pituitary gland and the optic chiasm. The surgical application of dextran was confirmed by comparison on section after β-galactosidase staining. After the fluorescent-dextran crystal application, the brains were maintained alive in the oxygenated physiological saline liquid as described (Oury et al., 2006, Science 313:1408-1413). After up to 16 hours of postoperative incubation, the brains were fixed in buffered 4% paraformaldehyde overnight at 4° C. and embedded into 3% agarose. All preparations were sectioned on a vibratome at 200 μm (FIG. 4B-C and 6F) or 50 μm (FIG. 11A) and analyzed under microscope (Leica).

### Example 13

### β-Galactosidase Staining

β-Galactosidase staining was performed on the tissues obtained from the Tph2+/− mice following standard procedures. Briefly, tissue samples were dissected after intracardial perfusion with ice-cold 4% paraformaldehyde in PBS, and fixed for 1-2 h. The samples were then washed three times with washing buffer (0.2% Nonidet P-40, 0.1% sodium deoxycholate, 100 mM phosphate buffer (pH 7.4), 2 mM magnesium chloride) for 15-30 min each and then stained at 37° C. overnight (12-16 h) in freshly prepared LacZ-staining solution containing 0.2% Nonidet P-40, 0.1% sodium deoxycholate, 100 mM phosphate buffer (pH 7.4), 2 mM magnesium chloride, 3 mM potassium ferricyanide, 3 mM potassium ferrocyanide, and 0.5 mg/ml X-gal (5-bromo-4-chloro-3-indolyl-D-galactopyranoside) protected from light. After the staining overnight, tissues were photographed before processing them for paraffin embedding for histology. Paraffin blocks were sectioned at 5-7 μm thickness, deparaffinized and counterstained with eosin, cleared in xylene, and mounted in DPX.

### Example 14

### μCT Analysis

Trabecular bone architecture of distal tibia was assessed using a micro computed tomography (μCT) system (VivaCT 40, SCANCO Medical AG, Switzerland). Tibial bone specimen was stabilized with gauze in a 2 ml centrifuge tube filled with 70% ethanol and fastened in the specimen holder of the μCT scanner. One hundred μCT slices, corresponding to a 1.05 mm region distal from the growth plate, were acquired at an isotropic spatial resolution of 10.5 μm. A global threshold technique was applied to binarize gray-scale μCT images where the minimum between the bone and bone marrow peaks in the voxel gray value histogram was chosen as the threshold value. The trabecular bone compartment was segmented by a semi-automatic contouring method and subjected to a model-independent morphological analysis (Hildebrand et al., 1999, J. Bone Miner. Res. 14:1167-1174) by the standard software provided by the manufacturer of the μCT scanner. 3D morphological parameters, including bone volume fraction (BV/TV), Tb.Th. (trabecular thickness), and connectivity density (Conn.D) were evaluated. The Conn.D is a quantitative description of the trabecular connection (Feldkamp et al., 1989, J. Bone Miner. Res. 4:3-11; Gundersen et al., 1993, Bone 14:217-222).

### Example 15

### Physiological Measurements

For food intake studies, mice were individually housed in metabolic cage (Nalgene, Rochester, N.Y.) and fed ad libitum. Food consumption amount was determined by weighing the powdered chow before and after the 24-hour measurement. Oxygen consumption (VO2) and respiratory exchange ratio (RER) were measured by indirect calorimetry method using a six-chamber Oxymax system (Columbus Instruments, Ohio). Mice were individually housed in the chamber and fed ad libitum. After 30-hour acclimation to the apparatus, data for 24-hour measurement were collected and analyzed as recommended by the manufacturers of the energy expenditure apparatus (Columbus Instruments, Ohio).

### Example 16

### Bone Histomorphometric Analyses

Bone histomorphometry was performed as previously described (Baron et al., 1983, Processing of undecalcified bone specimen for bone histomorphometry. In Bone histomorphometry: techniques and interpretation, R. R. Recker, ed. (Boca raton, CRC press), pp. 13-25; Chappard et al., 1987, Acta Histochem 81:183-190; Parfitt et al., 1987, J. Bone Miner. Res. 2:427-436). Briefly, lumbar vertebrae were dissected, fixed for 24 hr in 10% formalin, dehydrated in graded ethanol series, and embedded in methyl methacrylate resin according to standard protocols. Von Kossa/Von Gieson staining was performed using 7 μm sections for bone volume over tissue volume (BV/TV) measurement. Bone formation rate (BFR) was analyzed by the calcein double-labeling method. Calcein (Sigma Chemical Co., St. Louis, Mo.) was dissolved in calcein buffer (0.15 M NaCl, 2% NaHCO₃) and injected twice at 0.125 mg/g body weight on day 1 and 4, and then mice were killed on day 6. Four μm sections were cleared in xylene and used for bone formation rate (BFR) measurements. For the analysis of parameters of osteoblasts and osteoclasts, 4 μm sections were stained with toluidine blue and tartrate-resistant acid phosphatase (TRAP) respectively. Histomorphometric analyses were performed using the Osteomeasure Analysis System (Osteometrics, Atlanta, Ga.).

### Example 17

### Acute Dose Response of LY426965 in WT Mice

Animals were separated into individual cages one day prior to the experiment. Compound LY426965 was dissolved in water and fed orally (Gavage) according to the weight of the mouse at doses 1, 5, 10 and 20 mg/kg BW two hours prior to the commencement of the dark cycle. Control animals received the same volume of vehicle. Food intake was measured every 12 hours for 36 hours after giving one dose of the antagonist.

### Example 18

### Chronic Treatment of WT and ob/ob Mice with LY426965

In this experiment one-month old WT and ob/ob mice (housed individually in metabolic cages) were divided into different groups and injected with the LY426965 dissolved in water and diluted in saline (final concentration 0.9%) once a day, 2 hours prior to the commencement of the dark cycle, for 4 weeks. Body weight was recorded everyday and dose of the drug and vehicle adjusted accordingly. At the end of the experiment all mice were subjected to measurement of food intake every 12 hours over a period of 36 hours.

What is claimed is:

1. A method of treating an eating disorder associated with excessive weight gain, suppressing appetite, reducing body weight, or treating obesity in a patient in need of such treatment, comprising administering to the patient a therapeutically effective amount of one or more agents selected from the group comprising Htr1a receptor antagonists, Htr2b receptor antagonists, and Tph2 inhibitors or combinations thereof, including analogs, derivatives or variants thereof.

2. The method of claim 1, wherein the Htr1a antagonist is a member of the group comprising AP159; robalzotan; WAY100635; BMY 7378; piroxatrine; Rec 15-3079; DU-125530; lecozotan; indorenate; S-14489; S-15535; S-15931; SDZ 216-525; tertatolol; EF-7412; methiothepin; pindolol; LY426965, and compounds having the formula 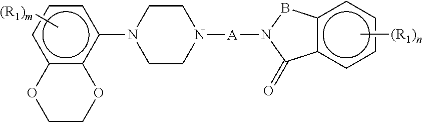
wherein R1 is halogen, lower alkyl or alkoxy, hydroxy, trifluoromethyl or cyano, m has the value 1 or 2 and n has the value 0 or 1,
A represents an alkylene chain containing 2-6 C-atoms which may be substituted with one more lower alkyl groups or a monocyclic (hetero)aryl group, and
B is methylene, ethylene, carbonyl, sulfinyl, sulfonyl, or sulfur; and
4-amino-2-(hetero)aryl-butanamides.

3. The method of claim 1, wherein the Htr2b antagonist is a member of the group comprising compounds having the formula: 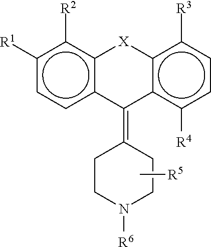
wherein R1 is selected from the group consisting of ethyl, propyl, isopropyl, butyl, isobutyl, pentyl, hexyl, ethoxy, propoxy, isopropoxy, butoxy, isobutoxy, phenoxy, trifluoromethyl, trifluoromethoxy, amino, dimethylamino, —CON(CH3)2 and —CON(C2H5)2;
R2 is a methyl, ethyl, propyl, isopropyl, butyl, isobutyl, pentyl, hexyl, hydroxy or hydrogen, or R1 and R2 together form a five-membered heterocycle, wherein a heteroatom in said heterocycle is an oxygen atom;
R3 is selected from the group consisting of methyl, ethyl, propyl, isopropyl, butyl isobutyl, pentyl, hexyl, hydroxy and hydrogen;
R4 is selected from the group consisting of hydroxy, methoxy, ethoxy, propoxy, isopropoxy, butoxy, isobutoxy, trifluoromethyl, amino, dimethylamino, diethylamino, fluorine, chlorine, bromine, methyl, ethyl, propyl, isopropyl, butyl and hydrogen;
R5 is methyl or hydrogen;
R6 is methyl or ethyl; and
X is S, N or Se;
provided that when R1 is ethoxy and X is S, at least one of R2, R3, R4 and R5 is not hydrogen; and
SB224289.

4. The method of claim 1, wherein the Tph2 inhibitor is p-Chlorophenylalanine or rifampin.

5. The method of claim 1 wherein the Htr1a receptor antagonist is an Htr1a-specific antagonist, and the Htr2b receptor antagonist is an Htr2b-specific antagonist.

6. The method of claim 1, wherein a reduction of the patient's pre-treatment body weight of at least 2 kg, at least 5 kg, at least 10 kg, at least 15 kg, or at least 20 kg; or a reduction of the patient's pre-treatment body weight of at least 3%, 5%, 10%, 15%, or 20% is achieved.

7. The method of claim 1, wherein the patient is administered an Htr1a and an Htr2b antagonist.

8. The method of claim 1, further comprising administering one or more Htr2c receptor agonists in an amount that increases or maintains the patient's pre-treatment bone mass, wherein the agonist is a member selected from the group comprising m-chlorophenylpiperazine, (+/−)-1-(4-iodo-2,5-dimethoxy-phenyl)-2-aminopropane; 1-(3-chlorophenyl)piperazine; desyrel; nefazodone; tradozone; 1-(alpha,alpha,alpha-trifluoro-m-tolyl)-piperazine; (dl)-4-bromo-2,5-dimethoxyamphetamineHCl; (dl)-2,5-dimethoxy-4-methylamphetamine HCl; quipazine; and 6-c35. hloro-2-(1-piperazinyl)pyrazine.

9. The method of claim 1, further comprising administering an amount of leptin or a leptin receptor agonist, or analogs, derivatives or variants thereof.

10. The method of claim 9, wherein the leptin agonist is LEP-(116-130) or a synthetic peptide corresponding to the sequence (Ser-Cys-Ser-Leu-Pro-Gln-Thr), or an analog, variant or derivative thereof.

11. A method for decreasing the weight gain in a patient taking an agent selected from the group comprising tricyclic antidepressants selected from the group comprising amitriptyline, imipramine, doxepine; selective serotonin reuptake inhibitors selected from the group comprising paroxetine and fluoxetine; irreversible monoamine oxidase selected from the group comprising phenelzine, isocarboxazid, tranylcypromine, and steroids, comprising administering one or more Htr1a receptor antagonists, Htr2b receptor antagonists, or Tph2 inhibitors or combinations thereof including analogs, derivatives or variants thereof in amounts that decrease the weight gained by the patient while taking the agent.

12. The method of claim 11, wherein the Htr1a antagonist is a member of the group comprising LY426965, AP159; robalzotan; WAY100635; BMY 7378; piroxatrine; Rec 15-3079; DU-125530; lecozotan; indorenate; S-14489; S-15535; S-15931; SDZ 216-525; tertatolol; EF-7412; methiothepin; pindolol; and compounds having the formula 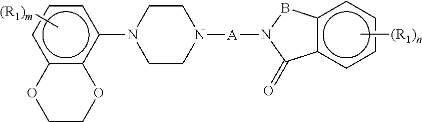
wherein R1 is halogen, lower alkyl or alkoxy, hydroxy, trifluoromethyl or cyano,
m has the value 1 or 2 and n has the value 0 or 1,
A represents an alkylene chain containing 2-6 C-atoms which may be substituted with one more lower alkyl groups or a monocyclic (hetero)aryl group, and
B is methylene, ethylene, carbonyl, sulfinyl, sulfonyl, or sulfur; and
4-amino-2-(hetero)aryl-butanamides.

13. The method of claim 11, wherein the Htr2b antagonists comprise compounds having the formula: 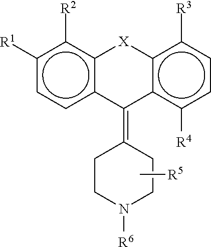
wherein R1 is selected from the group consisting of ethyl, propyl, isopropyl, butyl, isobutyl, pentyl, hexyl, ethoxy, propoxy, isopropoxy, butoxy, isobutoxy, phenoxy, trifluoromethyl, trifluoromethoxy, amino, dimethylamino, —CON(CH3)2 and —CON(C2H5)2;
R2 is a methyl, ethyl, propyl, isopropyl, butyl, isobutyl, pentyl, hexyl, hydroxy or hydrogen, or R1 and R2 together form a five-membered heterocycle, wherein a heteroatom in said heterocycle is an oxygen atom;
R3 is selected from the group consisting of methyl, ethyl, propyl, isopropyl, butyl isobutyl, pentyl, hexyl, hydroxy and hydrogen;
R4 is selected from the group consisting of hydroxy, methoxy, ethoxy, propoxy, isopropoxy, butoxy, isobutoxy, trifluoromethyl, amino, dimethylamino, diethylamino, fluorine, chlorine, bromine, methyl, ethyl, propyl, isopropyl, butyl and hydrogen;
R5 is methyl or hydrogen;
R6 is methyl or ethyl; and
X is S, N or Se;
provided that when R1 is ethoxy and X is S, at least one of R2, R3, R4 and R5 is not hydrogen; and
SB 224289.

14. The method of claim 11, wherein the Tph2 inhibitor is p-Chlorophenylalanine or rifampin.

15. The method of claim 11 wherein the Htr1a receptor antagonist is an Htr1a-specific antagonist, and the Htr2b receptor antagonist is an Htr2b-specific antagonist.

16. A method of treating an eating disorder associated with excessive weight loss, increasing appetite or increasing body weight, in a patient in need of such treatment, comprising administering to the patient a therapeutically effective amount of one or more Htr1a receptor agonists, Htr2b receptor agonists, or combinations thereof, including analogs, derivatives or variants thereof.

17. The method of claim 16, wherein the Htr1a agonist is [³H]-8-OH-DPAT (8-hydroxy-2-(di-n-propylaminotetralin) and the Htr2b agonist BW 723C86.

18. The method of claim 1, wherein the Htr1a antagonist is an anti-Htr1a antibody or fragment or variant thereof, and the Htr2b antagonist is an anti-Htr2b antibody or fragment or variant thereof.

19. The method of claim 16 wherein the Htr1a receptor antagonist is an Htr1a-specific antagonist, and the Htr2b receptor antagonist is an Htr2b-specific antagonist.

20. The method of claim 16, wherein an increase of the patient's pre-treatment body weight of at least 2 kg-20 kg or at least 3%-20%; or an increase of the patient's pre-treatment body weight of at least 3%, 5%, 10%, 15%, or 20% is achieved.

21. The method of claim 16, further comprising administering one or more Htr2c receptor agonists in an amount that increases or maintains the patient's pre-treatment bone mass.

22. The method of claim 11, wherein the Htr1a antagonist is an anti-Htr1a antibody or fragment or variant thereof, and the Htr2b antagonist is an anti-Htr2b antibody or fragment or variant thereof.

23. The method of claim 16, wherein the Htr1a antagonist is an anti-Htr1a antibody or fragment or variant thereof, and the Htr2b antagonist is an anti-Htr2b antibody or fragment or variant thereof.

24. The method of claim 21, wherein the Htr2c agonist is a member selected from the group comprising m-chlorophenylpiperazine, (+/−)-1-(4-iodo-2,5-dimethoxy-phenyl)-2-aminopropane; 1-(3-chlorophenyl)piperazine; desyrel; nefazodone; tradozone; 1-(alpha,alpha,alpha-trifluoro-m-tolyl)-piperazine; (dl)-4-bromo-2,5-dimethoxyamphetamineHCl; (dl)-2,5-dimethoxy-4-methylamphetamine HCl; quipazine; and 6-c35. hloro-2-(1-piperazinyl)pyrazine.

25. A method for achieving a desired level of appetite and bone mass in a patient, comprising administering one or more Htr1a or Htr2b receptor antagonists or agonists, and one or more Htr2c receptor antagonists or agonists, or analogs, variants or derivatives thereof in respective amounts that achieve the desired levels of appetite and bone mass

26. The method of claim 25, wherein the one or more Htr1a antagonists or Htr2b receptor antagonists or combinations thereof, and the one or more Htr2c receptor agonists are administered in respective amounts that reduce the patient's pretreatment level of appetite and increase the patient's pretreatment level of bone mass.

27. The method of claim 25, wherein the one or more Htr1a antagonists or Htr2b receptor antagonists or combinations thereof are administered in amounts that reduce or maintain the patient's pretreatment level of appetite, and the one or more Htr2c receptor antagonists are administered in amounts that reduce or maintain the patient's pretreatment level of bone mass.

28. The method of claim 25, wherein the one or more Htr1a agonists or Htr2b receptor agonists or combinations thereof are administered in amounts that increase or maintain the patient's pretreatment level of appetite, and the one or more Htr2c receptor antagonists are administered in amounts that reduce or maintain the patient's pretreatment level of bone mass.

29. The method of claim 25, wherein the one or more Htr1a agonists or Htr2b receptor agonists or combinations thereof are administered in amounts that increase or maintain the patient's pretreatment level of appetite, and the one or more Htr2c receptor agonists are administered in amounts that increase or maintain the patient's pretreatment level of bone mass.

30. The method of claim 25, wherein the one or more Htr1a agonists or Htr2b receptor agonists or combinations thereof, and the one or more Htr2c receptor antagonists are administered in amounts that reduce or maintain both the patient's pretreatment level of appetite and bone mass.

31. The method of claim 25, further comprising administering leptin or a leptin agonist or combinations thereof including analogs, variants or derivatives thereof to increase or maintain the patient's pretreatment appetite level.

32. The method of claim 25, further comprising administering a Tph2 inhibitor or analog, variant or derivative thereof to reduce or maintain the patient's pretreatment appetite level.

33. The method of claim 25, wherein the 5-Htr2B agonist is BW 723C86.

34. A method for increasing bone mass accrual in a patient having lower than desired bone mass by administering a therapeutically effective amount of a leptin receptor blocker, alone or together with an Htr2c agonist.
